(19) United States
(12) Patent Application Publication
(10) Pub. No.: US 2003/0100999 A1
(43) Pub. Date: May 29, 2003

(54) System and method for managing gene expression data

(76) Inventor: Victor M. Markowitz, Gaithersburg, MD (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 09/862,424

(22) Filed: May 23, 2001

Related U.S. Application Data
(60) Provisional application No. 60/206,571, filed on May 23, 2000.

(51) Int. Cl. G06G 7/48; G06G 7/58; G06F 19/00; G01N 33/48; G01N 33/50

(52) U.S. Cl. 702/20; 703/11

(57) ABSTRACT

The present invention pertains to a system and method for providing a common interface for multiple databases containing gene expression data, gene annotation data, and sample information in a relational format supporting efficient exploration and analysis.

### BACKGROUND OF THE INVENTION

1. Field of the Invention

The present invention relates generally to relational databases for storing and retrieving biological information. More particularly the invention relates to systems and methods for providing a common interface for gene expression data, gene annotation data, and sample information in a relational format supporting efficient exploration and analysis.

2. Description of the Related Art

DNA microarrays are glass microslides or nylon membranes containing DNA samples (e.g., genomic DNA, cDNA, or oligonucleotides) in an ordered two-dimensional matrix. DNA microarrays can be used to analyze gene expression and genomic clones or to detect single nucleotide polymorphisms (“SNP's”). The DNA used to create a microarray is often from a group of related genes such as those expressed in a particular tissue, during a certain developmental stage, in certain pathways, or after treatment with drugs or other agents. Expression of that group of genes is quantified by measuring the hybridization of fluorescently labeled RNA or DNA to the microarray-linked DNA sequences. By profiling gene expression, transcriptional changes can be monitored through organ and tissue development, microbiological infection, and tumor formation.

Also known as biochips, DNA microarrays can be created by linking monomeric nucleotides on the glass surface to make oligonucleotides. Another methodology, popular for making arrays of polymerase chain reaction (PCR) products and organismal genes, uses robotic instruments to spot thousands of DNA samples onto a surface. This high-throughput approach increases reproducibility and production.

Affymetrix of Santa Clara, Calif., provides high-volume production methods that can support the diagnostics or drug development industries. Affymetrix offers gene chip technology, which uses glass microarrays manufactured by a proprietary process that combines solid-phase chemistry and photolithography to build probes in situ. The glass wafers are packaged in plastic cartridges in which hybridization is carried out. Several hardware components form the gene chip suite. The gene chip fluidics station introduces the sample into the probe array cartridge. The Hybridization Oven processes up to 64 cartridges. Agilent Technologies designed its gene array scanner (monochrome; 20 μm resolution) to be used exclusively with Affymetrix microarrays, and the scanner is distributed by Affymetrix for integration into the gene chip suite. Affymetrix also offers a series of software solutions for data collection, conversion to GATC™ (“Genetic Analysis Technology Consortium”) database format, data mining, and a multi-user laboratory information management system (“LIMS”) system for power-hungry environments.

With today's DNA microarray technology one can easily collect large amounts of data to indicate what genes or SNP's are turned on or turned off during various disease states, following various pharmacological treatments, or following exposure to a variety of toxicological insults. However, this has resulted in a plethora of relational databases for storing and retrieving biological information, from which it is difficult for users to extract and compare information from the multiple databases.

Accordingly, there remains a need for a common interface for multiple databases containing gene expression data, gene annotation data, and sample information in a relational format supporting efficient exploration and analysis.

### BRIEF SUMMARY OF THE INVENTION

The present invention pertains to a system and method for providing a common interface for multiple databases containing gene expression data, gene annotation data, and sample information in a relational format supporting efficient exploration and analysis.

### BRIEF DESCRIPTION OF THE FIGURES

FIG. 1 presents the format for a first gene expression database relating to expression results, the GeneExpress expression result format.

FIG. 2 presents the format for a second gene expression database relating to expression results, the Expression Profiling Information and Knowledge System (EPIKS) expression result format.

FIG. 3 presents the format for a first gene expression database relating to samples, the GeneExpress sample format.

FIG. 4 presents the format for a second gene expression database relating to samples, the EPIKS sample format.

FIG. 5 presents the format for a first gene expression database relating to genes, the GeneExpress gene format.

FIG. 6 presents the format for a second gene expression database relating to genes, the EPIKS gene format.

FIG. 7 illustrates integrating multiple gene expression databases is to carry out an overall integration.

### DETAILED DESCRIPTION OF THE INVENTION

In one preferred embodiment of the present invention, there are multiple databases containing gene expression data, gene annotation data, and sample information in a relational format. However, if the gene expression data, gene annotation data, and sample information cannot be readily extracted, then the database does not provide for efficient exploration and analysis.

In a preferred embodiment of the present invention, the gene expression databases contain expression data for tens of thousands of genes, measured across thousands of samples. Scientific users need some means for selecting subsets of these data, analyzing them, and saving derived results for further analysis. The researchers may also want to combine and compare these derived datasets in various ways.

Over time, the users of the gene express database at a particular site will develop a large number of datasets, and will need a mechanism for organizing them so that related data are grouped together. Some data will be accessible to all users, some only to selected users, and some only to the user who created it. There is also a need for predefined read-only gene sets, selected according to commonly used criteria (for example, “all genes involved in the oxidative phosphorylation pathway”, or “all genes on rat chromosome 2”); these are preferably publicly available to all users.

Gene expression data by itself is of little value without some way of relating it to detailed information about the samples and genes from which it is drawn. Scientific users need an easy way to “drill down” from expression data to sample and gene details, extracted from our proprietary clinical data and from public data sources.

It is also difficult to obtain useful knowledge from gene expression data by examining tables of expression levels. The gene expression database users need a variety of visualization and analysis tools to clarify relationships between expression of different genes in specific tissues in different disease states. Some of the analysis tasks the gene expression database will be applied to include (1) finding genes that are consistently expressed or unexpressed in one organ or disease state but not in some other; (2) comparing expression level fold changes for a set of genes between two or more groups of samples; (3) grouping together genes whose pattern of expression is similar across a set of samples; (4) displaying a scatter plot of expression levels for a set of genes in a group of patients given certain medications, with the points colored according to the drug used, to highlight the genes upregulated or downregulated by the medications; (5) showing a graphical image of a metabolic pathway, with the expression levels of genes involved in the pathway indicated by colored spots; and (6) displaying the cytogenetic locations and expression levels of a set of differentially regulated genes on a chromosome map.

Some types of analysis—for example, clustering—generate lists of genes that are used later for further analysis steps. Users need to be able to maintain these resultant gene sets within the same data management framework as the source data, both for organizational convenience, and also so that they can track the “genealogical” information relating the gene sets and the source data.

Scalability is a potential stumbling block for external tools, because of the size of the datasets that can be generated with the gene expression database; memory-intensive operations such as clustering may require physical memory sizes on the order of hundreds of megabytes. The three-tier architecture proposed for the gene expression database circumvents this problem by centralizing memory- and compute-intensive operations on one or more middle-tier application servers.

In integrating multiple gene expression databases, there are several approaches to providing a common interface that permits efficient exploration and analysis.

With no integration, there is no change to any of the multiple databases. However, the query and analysis tools run would have to run separately on each of th multiple databases. In such circumstances, gene matching can be done only by means of sequence homology comparisons. Thus, one cannot analyze data from both databases together.

One approach to integrating multiple gene expression databases is to carry out the integration at the gene level. This may result in some of the multiple gene expression databases remaining unchanged. However, the query and analysis tools will run separately on the multiple gene expression databases, resulting in limited analysis of data from both databases.

A second approach to integrating multiple gene expression databases is to carry out the integration at the sample level. This may result in some of the multiple gene expression databases remaining unchanged. However, the query and analysis tools will run separately on the multiple gene expression databases, resulting in limited analysis of data from both databases.

A third approach to integrating multiple gene expression databases is to carry out an overall integration. This requires redesigning at least some of the multiple databases. The query and analysis tools are extended in order to run on new integrated database This results in tighter integration and uniform data analysis. The gene expression data is preferably analyzed via a run time engine (RTE). However, additional tools are needed, including data transformation and loading from the database to RTE.

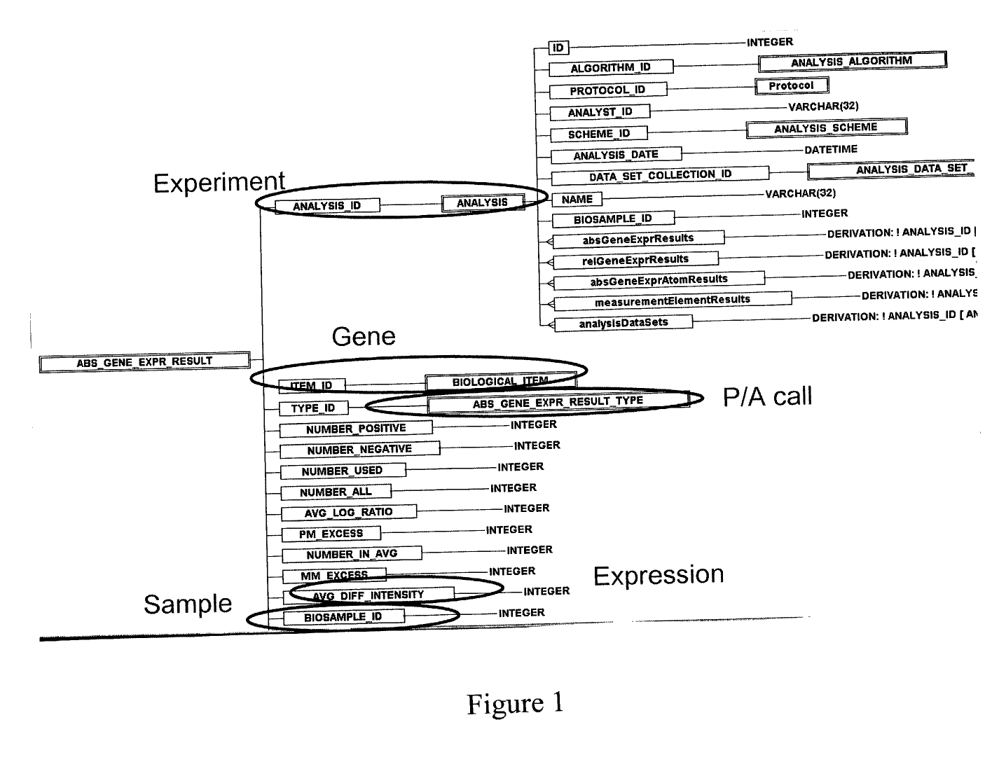

FIG. 1 presents the format for a first gene expression database relating to expression results, the GeneExpress expression result format.

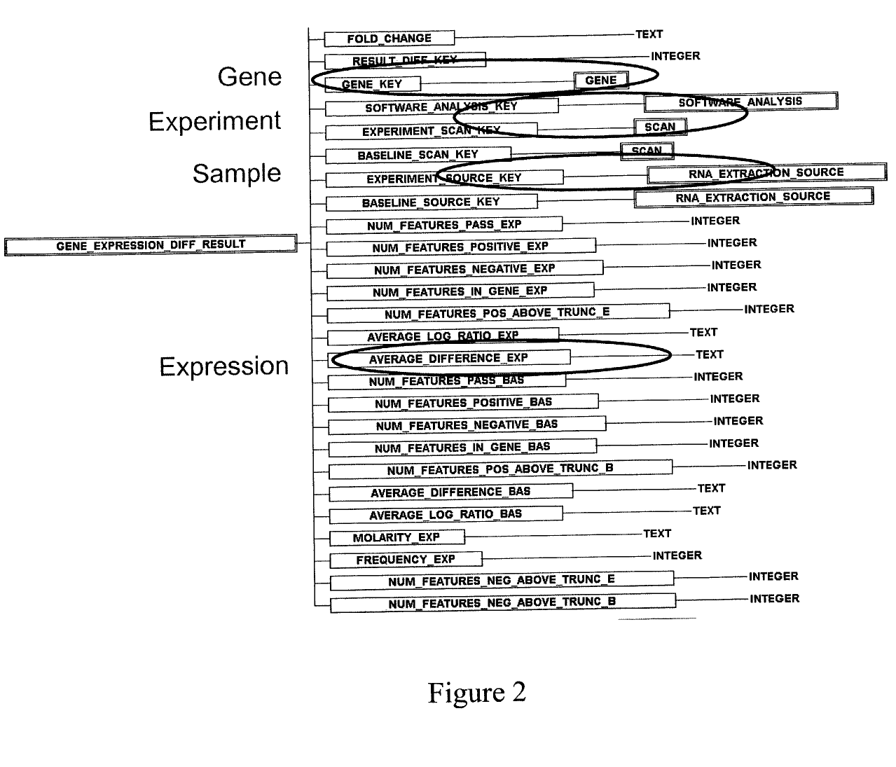

FIG. 2 presents the format for a second gene expression database relating to expression results, the Expression Profiling Information and Knowledge System (EPIKS) expression result format.

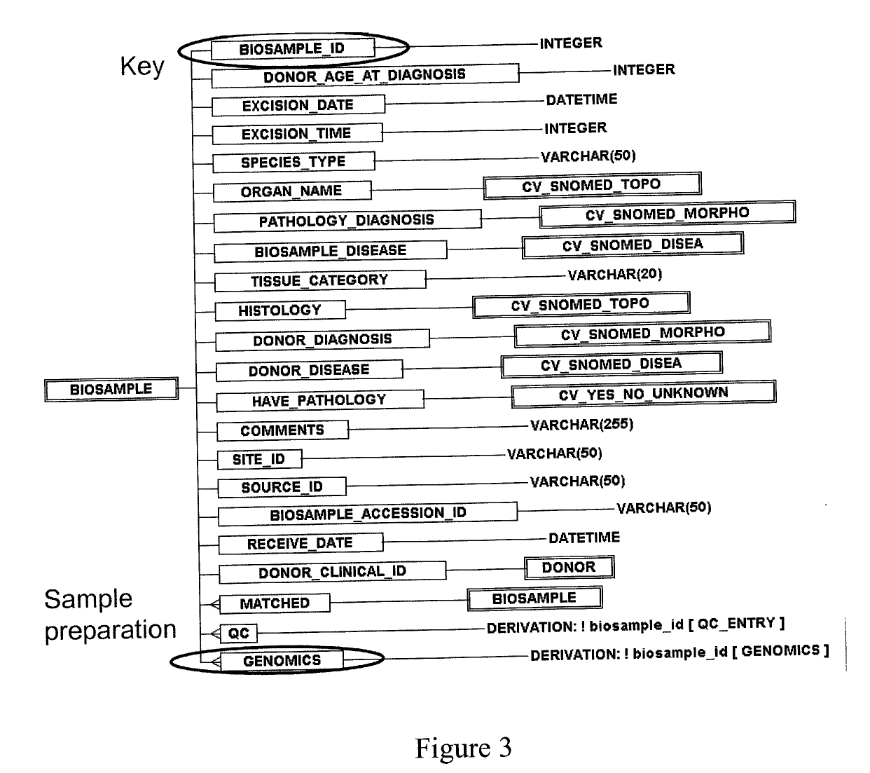

FIG. 3 presents the format for a first gene expression database relating to samples, the GeneExpress sample format.

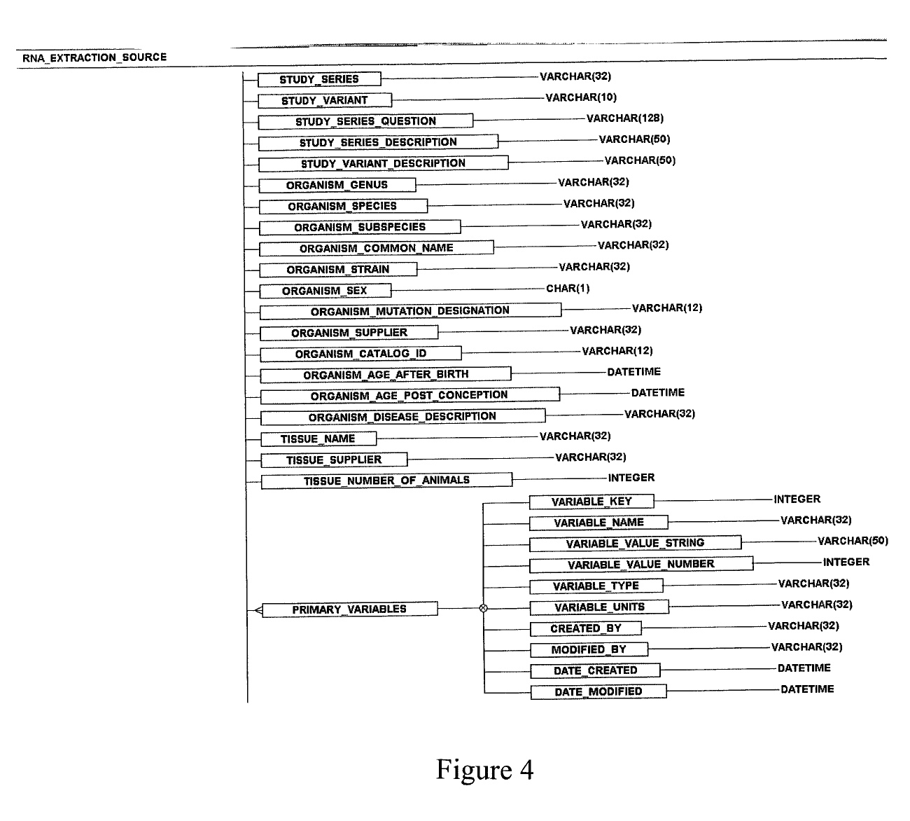

FIG. 4 presents the format for a second gene expression database relating to samples, the EPIKS sample format.

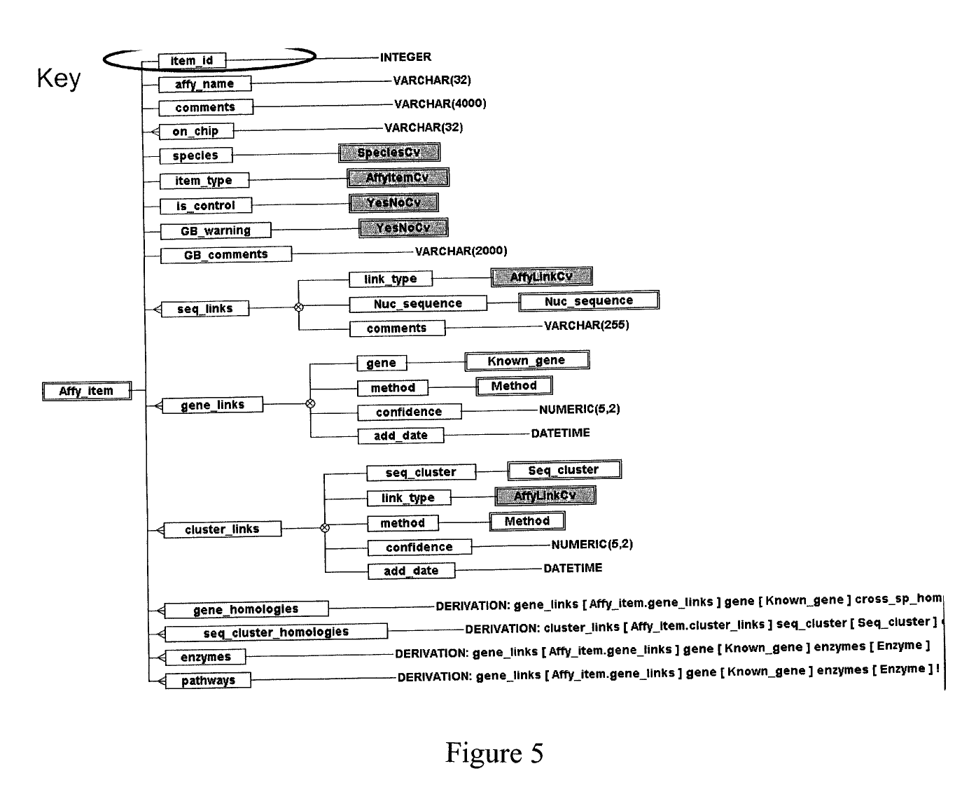

FIG. 5 presents the format for a first gene expression database relating to genes, the GeneExpress gene format.

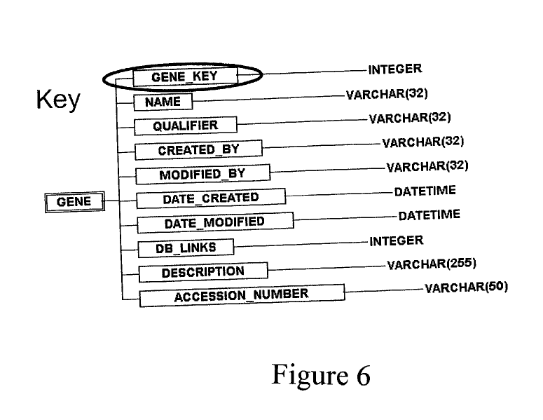

FIG. 6 presents the format for a second gene expression database relating to genes, the EPIKS gene format.

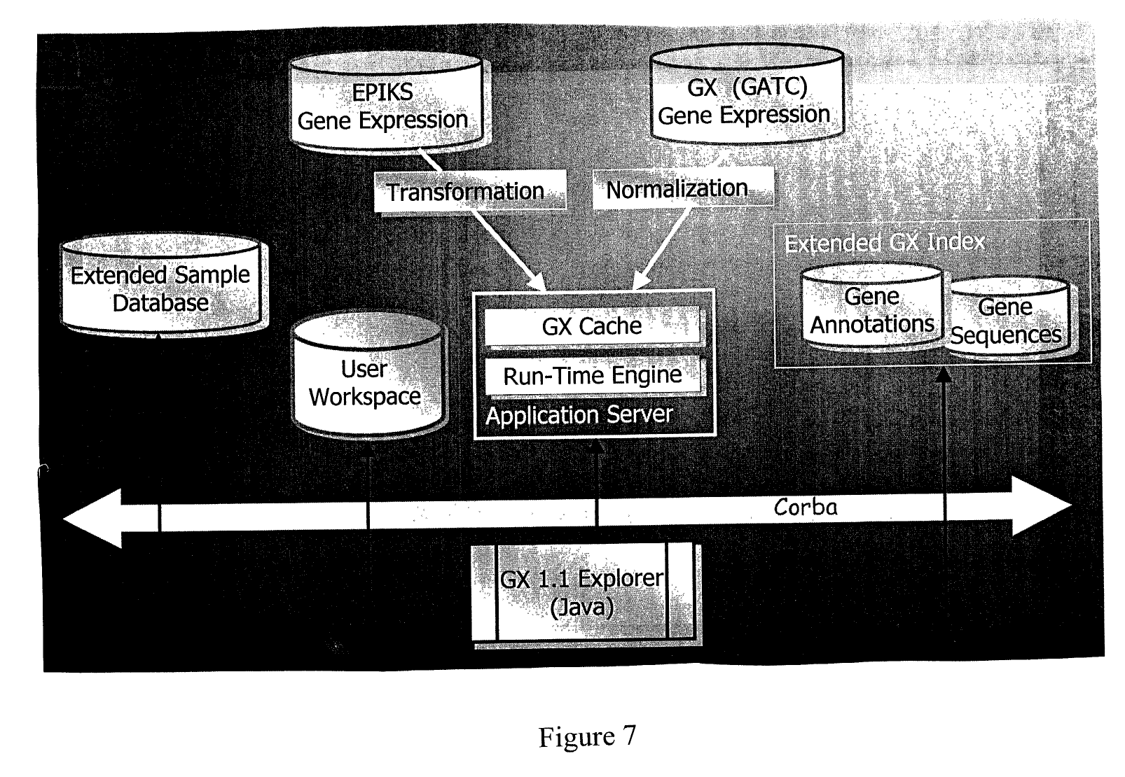

FIG. 7 illustrates integrating multiple gene expression databases is to carry out an overall integration. This requires redesigning at least some of the multiple databases. The query and analysis tools are extended in order to run on new integrated database This results in tighter integration and uniform data analysis.

In achieving this overall integration, the query and analysis tolls are preferably extended.

For example, in extending the gene index, one preferred aspect is to develop new classes and associations for the gene data in the database. These preferably include, but are not limited to, custom gene fragments and custom gene sequences, among others.

Another preferred aspect to extending the gene index is to develop new utilities for loading the existing data (and new data) into the developed new classes. Such an approach can allow for the preservatoion of custom associations in thew database previously developed.

Yet another preferred aspect to extending the gene index is to provide annotations for the gene data. Individual annotations for each gene or gene sequence can preferably be provided by OPM data entry tools. Custom gene fragment classification preferably rely upon sequence analysis (manual and/or automated) methods, which can be provided by sequence analysis workbench.

In loading the existing gene data into new classes, there are preferably several data tools which are employed, invcluding gene fragment classification and gene fragment annotation.

Gene fragment classification sorts gene fragments into two classes: (1) Known (with a GenBank accession number) and (2) Not known (not in GenBank). The Knwn classification employs name-based or sequence-based matching methods. The Not known classification utilizes sequence classification workbench to provide support for clustering with regard to a predetermined set of sequences; classification as novel/known/unknown sequence; association with public sequence clusters (Unigene, STACK); and periodic reclassification.

Gene fragment annotation permits automatic annotation for predefined fields, while allowing manual annotations of individual fragments. The manual annotations can preferably be provided by data entry tools.

Preferably, there are a number of methods employed in gene fragmernt classification. These include name based matching, sequence based matching, and manual data curation.

With regard to name based matching, gene fragments are associated with Unigene clusters. The Unigene clusters, in turn, are linked to Locus Link. Known genes are also associated with KEGG metabolic pathways.

Concerning sequence based matching, BLAST searches are employed to associate gene fragments with known genes and external sequences with Affymetrix fragments. In addition, with sequence based matching, gene sequence database sources are used to match; such database sources include gene sequences provided by Affymetrix and consensus sequences from Unigene or STACK clusters.

Regardng manual data curation, gene fragments without a Unigene match are reviewed to detect potential sequence data contamination.

For these reasons, the gene expression database preferably implements the most common analysis and visualization tasks internally, while allowing data to be exported to external applications.

The gene express database explorer interface (henceforth, “GX Explorer”) preferably consists of a main Workspace window and one or more query and analysis tool windows. The Workspace window provides project management functionality, and also acts as a launcher for the various tool windows. Tool windows are of several types, including, but not limited to, Sample Set, Gene Set, Gene Signature, E-Northern, Fold Change, Gene Signature Differential, Expression Data, and Cluster Analysis. Tool windows are preferably not displayed until the user explicitly launches them. A user can preferably display as many windows of each type as he/she wishes, subject to the memory limitations of his/her client PC.

The Workspace window preferably is a split pane window with menu and tool bars on top and a status/message area below. In one embodiment, the left-hand pane contains a tree view known as the Workspace Navigator, which displays the accessible folders and data objects in the Workspace Manager database. In this embodiment, the right hand pane shows the properties of the folders and objects displayed in the navigator, such as their type, ownership, permissions, and date of last modification.

Query and analysis tool windows are preferably laid out with tab panels, which allow the user to navigate between various tasks: specifying parameters, running queries or analyses, and different ways of visualizing results. Where appropriate, separate windows or split panes are used so that users can adjust parameters and see results immediately, without having to jump between panels.

The results of virtually any query or analysis method can preferably be exported to local files or external applications such as Excel, Spotfire, and S-Plus. These applications would run in separate external windows.

All windows preferably display the user's login name and the local time in a header area.

In the GX Explorer certain commands are generic, and can be accessed from the menubar of any window. Some of these will have toolbar equivalents. Menu and toolbar items are preferably enabled and disabled when appropriate. For example, in One preferred embodiment, within the File Menu commands include, but are not limited to Print and Exit. The Print command prints the currently displayed screen. In a window with multiple tabbed panels, only the currently active panel will be printed. Printouts are preferably labeled with the user's full name, the date, and the local time. The Exit command exits the GX Explorer application, can be invoked from any window, and displays a confirmation dialog with Yes and No buttons, where the user must click Yes to go ahead and exit.

The Edit Menu includes, but is not limited to Cut, Copy, Clear, Paste, and Select All. The Cut command deletes the currently selected data object, text, or other data, and puts it on the clipboard. The clipboard data must be accessible from external applications. Supported clipboard formats will vary according to what is selected. The Copy command copies the currently selected data object, text, or other data to the clipboard. The clipboard data must be accessible from external applications. Supported clipboard formats will vary according to what is selected. The Clear command deletes the currently selected data object, text, or other data. The Paste command pastes the contents of the clipboard at the “current” location. The meaning of “current location” depends on the context. The Select All command selects all rows in the display (visible or not), if a tabular display is visible in the active window.

The Export Menu contains items for each of the supported external file formats that GX Explorer can export data to: Excel, Spotfire, S-Plus, and Plain Text, preferably.

The Invoke Menu contains items for each of the supported external applications that GX Explorer can invoke and export data to, preferably, Excel and Spotfire.

The Window Menu is a dynamic menu, which contains an item for each of the active windows in GX Explorer, labeled with the window's title. When the user selects an item, the corresponding window is restored (if necessary) and brought to the top of the window stack. This is a useful navigational aid when the user has many windows active.

The Window menu also contains an item, “Arrange All”, which repositions the windows in a neat cascading stack starting from the top left corner of the desktop.

The Help Menu includes, but is not limited to, the Help System and the About commands. The Help System command brings up an index/search window for the help system. The About command displays a window showing the version numbers of GX Explorer, the various application servers (such as the Runtime Engine), and the schema and build versions of the GX Index, Sample DataBase and GX Data Warehouse databases.

Preferably, a user must have a username and password to access the Gene Express system. User accounts are created by an administrator using the interface described below; the administrator assigns the user an initial password, which the user can then change.

When the administrator creates a user account, he/she assigns the user to one or more groups. Groups are used as one means of controlling access to data objects; a user can make certain data objects accessible to other members of a group, that he/she would not want everybody else using the present invention to be able to see.

There is one special user that is predefined when Gene Express is installed, called “admin”. A user must know the admin password in order to use the administrative UI functions, such as creating user accounts. The admin user belongs to a predefined group, also called “admin”.

To access Gene Express, a user runs the Gene Express Explorer application (hereafter called GX Explorer), typically by double-clicking a desktop icon from Windows. GX Explorer first displays a login dialog, in which the user enters his/her username and password. The dialog provides a Help button, through which the user can obtain information such as how to obtain a Gene Express user account, the client system requirements, and how to log in.

When the user enters a valid usemame and password, the dialog is hidden and the main GX Explorer window is displayed.

The user administration operations are invoked through a separate Gene Express Admin application. A user can also change information in his/her own profile through the “Change Password” and “User Profile” commands in GX Explorer.

To start the Gene Express Admin application, the administrator runs Gene Express Admin from the Windows Start menu. A login dialog is displayed; the administrator enters the admin usemame and password. The main GX Admin window is then displayed.

The main window interface is simple; it contains tabbed panels for each of the main administration functions described below. There are also File, Edit and Help menus.

The Create User panel contains a form for creating new users. The form contains text fields for entering the user's usemame, password, full name, description, address, phone, fax, email address, and other contact info; and a multi-select listbox for entering the group memberships. The usemame, password, full name, and group membership must be entered; other attributes are optional.

The form contains buttons labeled “Create User” and “Clear Fields”. The Create User button creates a user with the specified attributes; Clear Fields resets the fields in the form to null or default values.

The Update User panel contains a form for updating information for existing users. The form contains a split pane view; the left hand pane contains list box allowing the administrator to select from a list of current usemames; the right pane contains text fields for the password, full name, description, address, phone, fax, email address, and other contact info; plus a multi-select listbox for updating the group memberships.

The form contains buttons labeled “Update” and “Delete User”. The Update button updates the attributes of the currently selected user; “Delete” deletes the currently selected user (after displaying a confirmation dialog).

The Create Group panel contains a form for creating a new user group and specifying its members. The form contains text fields for the group name and description, and a field picker widget for specifying the members. The left hand list box of the field picker lists all the valid users; the right hand list box shows the members selected to be in the group. The field picker contains Add and Remove buttons. The Add button adds users selected in the left hand list to the right hand list; the Remove button removes selected users from the right hand list. Double-clicking a user name in the left hand list is equivalent to selecting the user name and clicking Add; double clicking a user name in the right hand list removes the user from the list.

The form contains buttons labeled “Create Group” and “Clear Fields”. The Create Group button creates a group with the specified attributes; Clear Fields resets the fields in the form to null or default values.

The Update Group panel contains a form for updating the membership and description of an existing user group. The form contains a combo box for selecting the group name (containing a list of all the current groups), a text field for updating the group description, and a field picker widget for specifying the members. The left hand list box of the field picker lists all the valid users; the right hand list box shows the members selected to be in the group. The field picker contains Add and Remove buttons and supports double-clicking on users, as in the Create Group panel.

The form contains buttons labeled “Update” and “Delete Group”. The Update button updates the attributes of the currently selected group; “Delete” deletes the currently selected group (after displaying a confirmation dialog).

The Print function (accessed from the File menu) preferably supports printing a list of all current users, or all users from a selected group.

The Workspace window contains a tree view, called the Workspace Navigator, used to display and manage the data objects maintained by Gene Express. Data objects are displayed within project folders, represented by folder icons; the icons used to represent the data objects themselves vary and are used to indicate the object type. The look and feel is similar to that of the Macintosh Finder or the Windows Explorer; it resembles the former more than the latter in that folders and objects are displayed within the same tree view, so that the objects in multiple folders can be viewed simultaneously.

By default, data objects of all types are displayed under their parent folders. In all cases, the Workspace Navigator only displays the user's own folders and data objects, along with other users' folders and data objects that the user has read access to.

The tree view display is coordinated with the tabular display of folder and object attributes shown in the right hand pane of the GX Workspace window. Users can expand and collapse folders with the usual UI gestures (clicking the plus and minus icons or double-clicking the folder icons); the tabular display is preferably refreshed when this happens so that the attributes line up with the objects and folders they apply to.

The object attributes displayed include the owner of the object, the date and time it was created and last modified, its permissions, and its object type.

The Navigator tree view also appears as a component within dialogs, such as the one displayed when a user is prompted to select a Sample Set as input to a Gene Signature analysis. This is generically referred to as an “Open Data Object” dialog. The dialog is usually configured before it is made visible to only show data objects of a particular type—Sample Set, Gene Set, Gene Signature, etc.; the title bar also changes to “Save Sample Set”, etc., according to the type. The dialog also contains Open and Cancel buttons, whose function depends on where the dialog is displayed; clicking either one causes the dialog to be hidden.

The dialog can also be configured to show selected properties of the data objects shown. For example, an “Open Sample Set” dialog may display the number of samples in each sample set listed. This is useful when the user is selecting a sample set for input to a Gene Signature analysis.

In situations where the user needs to save a data object in the Workspace Manager database (e.g., when creating a Sample Set or Gene Set), the user is prompted for information about the new object through a “Save Data Object” dialog. This dialog also contains a Workspace Navigator tree view; here it is used mainly to select the folder where the new data object will be saved. (Depending on the situation, the user may be allowed to select and thereby replace an existing data object.) Only the user's own folders and objects within them are displayed; objects of the wrong data type are always grayed out; if the user is not allowed to overwrite objects, all objects are grayed out.

The dialog also contains a text area, where the user can enter an optional description; a series of combo boxes, for setting the group and world access permissions (defaulting to “no access”; and “Save” and “Cancel” buttons. When the user clicks the Save button, the object is created or replaced; if he/she clicks the Cancel button, no changes are made to the Workspace Manager database; if he/she clicks either one the dialog is hidden.

Note that the group permissions interface differs from that in the WebUI, in that it supports different permission levels for different groups. Therefore, instead of a single combo box that controls the permissions for all the groups the user belongs to, the dialog needs to display a table or grid of group names and permission combo boxes, with one row for each group the user belongs to, plus a row for the world permissions.

As an aspect of project management, Gene Express enables users to organize data objects into project folders, which are represented using folder icons in the Navigator. Each user has his/her own top-level folder, with the same name as his/her Workspace Manager username. It may be subdivided into project-specific folders. There is a predefined user named “Public”, whose folders contain various read-only data objects predefined by Gene Express. These read-only objects may be copied into a user's own folder, and the copies may be edited. Folders are assigned permissions, so that other users or groups of users may be granted access; by default folders are not accessible by anyone other than the user who created them.

Users can perform multiple operations on project folders. Except where indicated, users invoke these operations by either (1) right-clicking on the folder icon, which brings up a popup menu, and selecting the appropriate item from the menu; (2) left-clicking on the folder icon, which causes it to be selected and highlighted; and then choosing the appropriate item from the File menu on the Workspace window menubar; or (3) left-clicking to select the folder icon, then clicking a button in the Workspace window toolbar (if one is provided for the given operation).

The Create Folder command creates a new empty folder as a subfolder of the selected folder. The folder is initially displayed in the Navigator tree view with an editable text field in place of its label; the user must enter a name for the folder and press Enter. Names may contain any characters except the “/”, which is reserved as a pathname separator. If the user presses Enter without entering a name, an alert dialog is displayed informing the user that he/she must enter a name. If the name is the same as that of an existing subfolder of the same folder, the user is warned to enter a different name.

The folder is initially created with “no access” permissions for the world and for the user's group.

The Rename Folder command temporarily replaces the folder's name label with an editable text field, in which the user enters the new folder name. When the user presses the Enter key, the folder is renamed and the label is redisplayed with the new name.

If feasible, users are preferably able to invoke this operation using the standard Windows and Macintosh gesture, i.e. single-clicking on the folder label and then not moving the mouse for a specified time delay.

Set Permissions

The Set Permissions command presents a dialog with a table or grid of group names and permission combo boxes, with one row for each group the user belongs to, plus a row for the world permissions.

The left hand column gives the names of each group the user belongs to, plus “all others” for the world permissions; the right column contains combo boxes with which the user selects the permissions for that class of users. For each class of users, the user can select from “No Access”, “Read Only” and “Full Access”.

Set Permissions can be invoked for multiple folders simultaneously, by first control-clicking their icons in the Workspace Navigator to select them.

The Delete Folder command deletes the selected folder or folders and refreshes all Workspace Navigator views. A confirm dialog is displayed, allowing the user to cancel the operation if he/she changes his/her mind. The deleted folders are actually moved to the user's “Trash” folder, and eventually removed according to a FIFO scheme; until then the user can retrieve them by moving them to another folder.

The Copy Folder command makes a recursive copy of a folder and its contents under another parent folder. A folder cannot be copied to itself.

The user can invoke this operation in the multiple ways. The first is by dragging the folder to be copied using the right mouse button and dropping it on the destination folder. Because the right button was used for dragging, a popup menu is displayed containing the items “Move”, “Copy” and “Cancel”, with “Move” (the default operation) highlighted. The user then selects “Copy” to force a copy instead. The UI must provide the proper feedback so that a “stop” cursor is displayed when the folder cannot be dropped at the current cursor location. The second way is by selecting the folders to be copied, copying them to the clipboard, selecting the destination folder, and performing a Paste operation. Multiple folders can be copied by this method.

The Move Folder command places a folder under a different parent folder. A folder cannot be moved to itself or one of its children. The user can invoke this operation in the multiple ways, including dragging the folder to be copied using the left mouse button and dropping it on the destination folder. The UI must provide the proper feedback so that a “stop” cursor is displayed when the folder cannot be dropped at the current cursor location. Another method to move a folder is by selecting the folder to be moved, performing a Cut operation to put it on the clipboard, selecting the destination folder, and performing a Paste operation.

Within the present invention, there are several types of data objects: Sample Sets, Gene Sets, and the various kinds of analysis objects (Gene Signature, Fold Change, among others).

A SampleSet represents a list of human, animal and cell line samples from the same species, for which clinical and/or gene expression data are available in the Sample DB and the GX Data Warehouse. A user defines a SampleSet through one of several query methods. The internal representation of a Sample Set is a list of “genomics IDs”, which are numeric identifiers between 1 and 99999 used by the Genomics Production group to track samples. The ordering of samples may be important, and so must be preserved by the various query and analysis operations.

Note that species is a separate distinction from sample type. In the WebUI Sample Sets are restricted to contain samples of the same type, because different sample types are associated with different attributes. In GX Explorer the mixed type restrictions (but not mixed species) are removed, since tissue and cell-line samples are run on the same chip types; users may want to include data from both in an analysis.

A Sample Set may be defined by the results of a database query at a particular time. It is important to remember that the list of samples in the set does not change when new samples are added to the GX Data Warehouse satisfying the original query criteria; that is, the query is not persistent.

Users may edit the contents of a Sample Set in various ways, including by displaying the Sample Set in a tabular view with editing enabled, and deleting individual samples or by by performing a set intersection, set union, or set difference operation with another Sample Set.

Users may select any set of sample attributes to be displayed when a Sample Set is visualized or exported. The list of sample attributes is stored as part of the user's preferences, so that the attributes don't need to be reselected every time the user displays a Sample Set. Separate lists of sample attributes are maintained for the various sample types (human, animal and cell line).

A Gene Set represents a list of gene fragments, each on a specific Affy chip type, selected according to user-supplied criteria. A user defines a Gene Set by performing one of various types of queries against the Gene Express Fragment Index (a.k.a. the GX Index). The internal representation of a Gene Set is a list of gene IDs, which are combinations of the numeric Affy item_ids for each fragment with the two digit IDs of the Affy chip containing each fragment instance. The chip IDs are required to distinguish between cases where the same Affy gene fragment is tiled on more than one chip type; there are several hundred such cases.

The ordering of gene fragments may also be important, and so must be preserved by the various query and analysis operations.

A Gene Set is initially defined by the results of a query against the GX Index at a particular time. The data in the GX Index change over time; not only are new chip types released by Affymetrix and used to generate expression data for Gene Express, but also new information is generated linking known genes and EST clusters to existing Affy gene fragments. Thus the same query run at a later time may return a different list of gene IDs. It is important to remember though that these changes in the data content of the GX Index will not affect the list of gene IDs in existing Gene Sets.

Users may edit the contents of a Gene Set in multiple ways, including by displaying the Gene Set in a tabular view with editing enabled, and deleting individual Affy fragments or by performing a set intersection, set union, or set difference operation with another Gene Set.

Users may select a list of gene attributes to be displayed when a Gene Set is visualized or exported, from a predetermined set of cached attributes: gene symbol, gene name, sequence cluster name, cytogenetic band location, chip name, GenBank ID,and pathway list. The list of gene attributes is stored as part of the user's preferences, so that the attributes don't need to be reselected every time the user displays a Gene Set. The set of cached attributes the user can choose from may expand in future releases; arbitrary attributes cannot be queried from the GX Index database for performance reasons.

The Workspace Manager supports other types of data objects which are associated with the various analyses that can be run in Gene Express: Gene Signatures, Gene Signature Differentials, Fold Change Analyses, Electronic Northems, and Cluster Analyses. The user can perform all the standard workspace operations on these objects (copy, move, rename, etc.), or can load the object for visualization or further analysis. Although there may be situations where the actual data results are stored with these objects, usually the Workspace Manager only stores the information necessary to rerun the analysis (e.g., the Sample Set and thresholds for a Gene Signature).

The process for creating an analysis data object varies according to the type of analysis method. Generally the user is given the option, after running an analysis, to save the analysis in the workspace. The user enters a name, description and initial permissions, and selects a folder in which the analysis object will be saved.

A user can perform most operations on existing data objects by left-clicking their icons in the Workspace Navigator to select them, and then choosing a menu or toolbar item. Some operations can be performed on multiple objects at once; the user selects multiple objects by control-clicking them. There are two kinds of menus in the GX Explorer:

Pulldown menus, activated from the main menubar. Most of the data object operations are found in the File menu (even though data objects are stored in the workspace manager database rather than in files).

Popup or “contextual” menus, activated by right-clicking one of the selected objects in the Navigator.

Some operations only apply to certain types of data objects. The corresponding menu and toolbar items will be enabled or disabled according to the type of objects selected. If multiple objects are selected, only the items for operations that can be performed on multiple objects will be enabled.

The Open operation loads one selected object for visualization and (if appropriate) for editing. This operation is highly polymorphic and will display different information for different data object types. Generally, when the user selects this operation, GX Explorer will create a new window of the appropriate type (a Sample Set window for a Sample Set, a Gene Signature window for a Gene Signature, etc.) and open it to a summary view of the object. The contents of the summary view will vary according to the object type, but will usually be the first view displayed after a query or analysis is run.

Double clicking an object in the Workspace Navigator opens it, as does selecting the object and then choosing Open from a menu.

The Move operation moves the selected data object into a different folder. The user must have write access to both the source and destination folders.

The user can invoke the move operation in multiple ways, including (1) dragging the object to be copied using the left mouse button and dropping it on the destination folder. The UI must provide the proper feedback so that a “stop” cursor is displayed when the object cannot be dropped at the current cursor location and (2) selecting the object to be moved, performing a Cut operation to put it on the clipboard, selecting the destination folder, and performing a Paste operation.

The Copy operation creates a copy of the selected data object. There are two versions of this operation. The first is in-place copy where the user selects an object, then chooses the Copy menu or toolbar item. This copies the object to the clipboard. The user then chooses the Paste menu or toolbar item; this creates a copy of the object in the same folder, but with “copy” appended to the name of the object. The second is copy from another folder. The usual way to perform this is via drag-and-drop, using the right mouse button. When the mouse button is released over a legal destination folder, a popup menu is displayed containing the items “Move”, “Copy” and “Cancel”, with “Move” (the default operation) highlighted; the user then selects “Copy” to force a copy instead. The UI must provide the proper feedback so that a “stop” cursor is displayed when the object cannot be dropped at the current cursor location.

The user can also copy the object to the clipboard as for an in-place copy, and then select a destination folder and perform a Paste operation. The Paste will fail if the destination folder is not writeable by the user.

The Rename Data Object operation temporarily replaces the object's name label with an editable text field, in which the user enters the new object name. When the user presses the Enter key, the object is renamed and the label is redisplayed with the new name.

Users are preferably able to invoke this operation using the standard Windows and Macintosh gesture, i.e. single-clicking on the object label and then not moving the mouse for a specified time delay.

Permissions

The Permissions operation presents a dialog with a table or grid of group names and permission combo boxes, with one row for each group the user belongs to, plus a row for the world permissions. The left hand column gives the names of each group the user belongs to, plus “all others” for the world permissions; the right column contains combo boxes with which the user selects the permissions for that class of users. For each class of users, the user can select from “No Access”, “Read Only” and “Full Access”.

The Permissions operation can be invoked for multiple objects simultaneously, by first control-clicking their icons in the Workspace Navigator to select them.

The Delete Object operation deletes the selected object(s). A confirmation dialog is displayed, allowing the user to cancel the operation if he/she changes his/her mind. The deleted objects are actually moved to the user's “Trash” folder, and eventually removed on a FIFO basis; until then the user can retrieve them by moving them to another folder.

A number of operations are accessed through the “My Profile” menu in the Workspace window. These include, but are not limited to, Change Password and User Profile.

The Change Password operation presents a dialog with three password fields, in which the user enters his/her old and new passwords; the new password is entered a second time for confirmation. The text entered is hidden or echoed as “*” characters. “OK”, “Cancel” and “Help” buttons are provided; the Help button provides information on the required and recommended length and content of password strings.

The User Profile operation presents a form in which the user's profile information are displayed and, in some cases, can be edited. The editable information includes the user's name, description, address, phone, fax, email address, and other contact info; the list of groups the user belongs to is displayed but is not editable.

A user creates a new Sample Set in one of multiple ways. The first is from the Queries menu in the menu bar by selecting “Sample Set”; this brings up a Sample Set window with the “Search Parameters” tab selected. The second is from the Samples menu in the menu bar by selecting “Genomics ID Query”; this brings up a Sample Set window, with the “Genomics ID Query” tab selected. This interface is primarily used by internal Gene Logic users.

A second way is by selecting an existing Sample Set in the Navigator, duplicating it (by selecting Copy from the File menu or contextual menu, and then pasting the Sample Set in a different folder), and then opening it. This brings up a Sample Set window with the tabular display tab selected, listing the samples and their attributes in a tabular display. The user can remove samples from the list if he/she wishes.

The Sample Set and Gene Set windows use a common query interface. In both cases, the objective is to find OPM objects of a specific OPM class.

An OPM class search searches and/or displays objects of a specific OPM class. A user performs one of these searches from a Sample Set or Gene Set window. There may be multiple such windows open at a time.

The operations involved in an OPM class search are search construction, running the query and viewing the results, and saving and loading the results in a result set. In search construction, the user can specify the search conditions, specify the returned attributes, and specify the order of the results. In running the query and viewing the results, the results may be viewed in a tabular display or in a form display. In saving and loading the results in a result set, the user can save the results as, for example, a sample set or a gene set.

The Search Construction interface is a tabbed panel (labeled “Search Parameters”) in a Sample Set or Gene Set window. It contains a split pane view, with an attribute selector on the left that shows the display names for all sample or gene attributes, grouped hierarchically in a tree view. In the right pane, there are three tabbed subpanels, one for each of the sub-operations of search construction: entering conditions, selecting display attributes, and specifying sort criteria. On the bottom, there is a footer panel containing “Search”, “Add” and “Remove” buttons, which is shared by the three sub-operations. The attribute selector is also shared; however, the user can select the same or different attributes for each of the three sub-operations.

After a search, the user may return to the search construction panel, alter the conditions, and rerun the specified search.

The display attributes and the sort-by attributes are saved in the user's preferences.

The Sample Set window has an additional pull-down menu, Species, which is not present in the Gene Search window. The pull-down menu requires the user to choose between the possible species values in the Sample DB. The value set in the species menu is saved in the user's preferences. Changing the selection in the species menu preferably resets the Search Parameters tree and clear the Conditions list. Note that the choice of the species preferably affects whether certain classes and attributes are present in the Search Parameters tree. For example the [Donor Animal] class preferably is not present if the species is Human, and the [Donor Human] class is not present if the species is rat or mouse. However, the [Donor Cell-Line] class may be present for any species, together with the other appropriate Donor subclass, since cell line and tissue samples may be mixed in a Sample Set. The search interface code preferably detects ANDs of constraints on attributes from different Donor subclasses and prevent the user from executing the query if they don't make sense.

Sample searches are constrained by another implicit condition, which is the ownership of the sample data. An instance of the Gene Express data warehouse may contain private data from samples provided by several external alliance partners. One alliance partner preferably is not permitted to see another partner's private data. Since all analysis operations and data exports require the user to specify a SampleSet, external users are restricted from having access to another alliance's data by only returning samples in a Sample Search that are either public or owned by the alliance partner.

There may be a many-to-one correspondence between user groups in the Workspace Manager and alliance partners, since each partner may want to separate its users into distinct groups; the alliance name may need to added as an attribute of the user group to enable ownership filtering.

To select the attributes and values used to specify query conditions, the user clicks the Conditions tab to display the Conditions tab panel. The Conditions panel contains two radio buttons labeled “all of the following” and “any of the following”, and a sub-pane containing a list of conditions. Each condition consists of an attribute name followed by one or more components. Some conditions may be spread over several lines, in which case the components of the condition are aligned in columns, with the first column containing the attribute name in the first line and being blank in the later lines.

To add conditions to the condition list, the user selects (clicks on) each attribute in the attribute tree view, and then clicks the “Add” button; or double-clicks the attribute in the tree. To remove conditions from the list, the user selects them in the list and clicks the “Remove” button, or double-clicks them in the list.

Each time the user adds an attribute to the conditions list, the program will add a line to the Conditions panel consisting of the attribute name followed by one or more fields and zero or one button. The fields allow the user to assert conditions on a value of the attribute. The buttons allow multiple values to be specified for a condition. The type of fields used to enter these conditions depends on the attribute's type and on additional configuration information, as follows:

For most attributes, the first field displayed is a picklist, used to select a query operator. The list of operators the user can choose from depends on the attribute type, as follows:

Numeric: equals, does not equal, less than, less than or equal to, greater than, greater than or equal to, between, not between

String: matches, does not match, starts with, ends with, contains, does not contain

Date: before, after, between, not between

Abstract: is, is not, plus string operators (applied to ID attributes)

Any: is null, is not null

In addition certain operators will be grayed-out or omitted from the pick-list for certain kinds of attributes, as follows:

is null and is not null are grayed-out for any non-optional attributes, or paths ending in a non-optional attribute.

not equals to, less than, not less than, more than, not more than, not between, not matching and is not are preferably be grayed-out for any multi-valued attribute or path.

Case sensitivity of string matches is controlled from the Options menu of the search window, applies to all searches, and is saved in the user's preferences.

Zero, one or two fields are displayed to the right of the operator picklist for entering the condition values. The number of fields depends on the query operator selected. If the query operator is changed to one requiring a different number of fields or fields with different types, then the fields will be cleared and redisplayed. However if the query operator is changed to another operator requiring the same number and type of fields, then any values typed or picked in the fields will be preserved.

The “between” and “not between” operators require two fields; “is null” and “is not null” don't require any; all others require just one.

The type of value fields depends on the attribute type, as follows:

String and numeric: Text field. Scientific notation is preferably supported for numeric value entry, if possible.

Date: Calendar field. These are text fields with a calendar icon button next to them; clicking the icon displays a calendar widget from which the date can be selected. The date format is preferably configurable from the Options menu, and saved in the user's preferences.

Abstract: Combo box or field. If the attribute has the OPM_UI_WIDGET property set to “List” in the OPM schema file, a combo box will be displayed, containing a list of all distinct values for the attribute in the database, represented by their ID attribute values. If not, a field widget is supplied, in which the user can enter all or part of an ID attribute value; the query operator will then be a match operation, which is then applied to the ID attributes of the instances of the abstract attribute's class.

Certain primitive attributes may also have the OPM_UI_WIDGET property set to “List” in the OPM schema; values for these may be selected from combo boxes, displaying a list of all the distinct values returned for the attribute from a database query. For example, the BIOSAMPLE.SITE attribute is one of these attributes. The combo box is editable, so that the user may enter a partial string match and use one of the match operators to enter the condition.

Note that, unlike in the WebUI, values displayed in a combo box list are not restricted by any preceding search conditions.

Zero or one buttons may be displayed to the right of the last field in the condition. Whether a button is displayed, and the label of the button depends on the operator chosen in the operator picklist:

Equals, matches, starts with, ends with, contains, is: A button is displayed with the label OR.

Does not equal, does not match, does not contain, is not: A button is displayed with the label AND.

All other operators: No button is displayed.

If the operator for a condition is changed using the operator pick-list, then the button for the condition may be added, removed or relabeled, depending on the choice of operator.

Some attribute types get special treatment. Values for abstract attributes of type CV_SNOMED_TOPO are selected from a tree view, reflecting the hierarchical nature of the SNOMED vocabulary; if the user selects a non-leaf node in the tree, the search will match any vocabulary term in the selected subtree. For example, if the user selects the parent node for “BRAIN”, the corresponding condition will match all values that are grouped under BRAIN. For these attributes, a query operator field is not supplied; an operator is already implied in the translation of the query condition (which looks something like “@topo.TERMCODE LIKE ‘T-A0%’”).

The SNOMED topography vocabulary contains many terms such as “BRAIN, NOS”, where “NOS” stands for “not otherwise specified”; the “,NOS” is preferably stripped from these terms in the SNOMED tree view, since users find it confusing.

Conditions for which the user enters blank values will not be used for the search.

If a condition ends in an AND or an OR button, then the condition may be extended with additional values. To extend a condition the user preferably clicks on the AND/OR button. Each time this is done the following changes to occur to the conditions list panel:

A new line is added immediately after the last line of the condition. The new line consists of one or two fields (depending on the condition operator) of type dependant on the type of the attribute (as above). The fields of the new line are aligned with the corresponding fields of the previous line, but the new line does not contain the attribute name or operator pick-list.

The AND/OR button is moved vertically to align with the middle line of the condition (or between the middle to lines if the number of lines is even). A large right-hand curly bracket “}” is drawn between the right-most field and the button, so that the curly bracket spans the height of the condition.

In order for a condition to be extended in this way, all of the fields of the previous lines preferably have values in them. Otherwise the AND/OR button is disabled.

Each operator represents a relationship between attribute values and zero, one or two values input (dependent on the number of fields following the operator).

For example the matches operator defines a string matching relationship between attribute values and single string values—the second string value may contain the wild-card character ‘%’ which matches any sub-string. The other operators have similar obvious relationships associated with them.

A single-line (non-extended) condition is satisfied by an object if one of the attribute or path values of the object is in the appropriate relationship with the values input by the user in the condition fields.

Note that, if an attribute or path is multi-valued, then, in order to satisfy a condition, it is only necessary for one value of the attribute or path to be in the relationship with the field value. For example, it a user specified the condition Comments matches “%foobar%”, then this condition would be satisfied by any object with a Comments attribute containing at least one string containing the sub-string “foobar”. It would not require an object to have “foobar” as a sub-string of all its Comment values.

A multi-line (extended) condition with an OR button (i.e. with one of the operators equals, matches, starts with, ends with, contains or is) is satisfied by an object if one of the attribute or path values of the object is in the appropriate relationship with at least one of the values input by the user into the condition fields.

A multi-line (extended) condition with an AND button (i.e. with one of the operators does not equal, does not match or is not) is satisfied by an object if the attribute or path value of the object is in the appropriate relationship with all the values specified by the user in the condition fields.

When multiple conditions are entered in the Conditions panel, they are combined either with a boolean AND operation or a boolean OR operation. In the first case, the results of a search will be those objects which satisfy ALL of the conditions in the conditions panel, while, in second case, the results will be those objects which satisfy AT LEAST ONE of the conditions in the query panel.

The top of the conditions panel contains a label, “Find samples” (or “Find gene fragments”), followed by a radio button group with options “Matching all of the following” (the default) and “Matching any of the following”. If the “all” button is selected then the conditions will be combined using a boolean AND operator, while if the user selects the “any” button instead, the conditions will be combined using an OR operator. This does not affect how the individual lines of a multiple-line condition are combined.

The bottom of the Conditions panel may contain a paraphrase of the current list of conditions, e.g. “You selected Gender=Male AND Pathology Diagnosis contains ‘carcinoma’”.

To select the attributes that will be displayed when the sample or gene search is executed, the user chooses “Select Display Attributes” from the View menu to bring up the Select Attributes dialog. The dialog contains a list of the attributes currently selected, in the order they will be displayed in a tabular or form view. There are also “Add” and “Remove” buttons.

To add attributes to the display list, the user selects (clicks on) each attribute in the attribute tree view ,and then clicks the “Add” button; or double-clicks the attribute in the tree. To remove attributes from the display list, the user selects them in the list and clicks the “Remove” button, or double-clicks them in the list. To change the order of attributes, the user drags them upward or downward in the list.

The condition attributes are automatically included in the display list; the user can remove them if desired.

To select the attributes that will be used to sort the samples or genes returned by the search, the user clicks the Order By tab to display the Order By tab panel. The Order By panel includes two columns for each attribute. The first column contains a list of the attributes currently used for sorting, with the primary sort attributes displayed first. The second column displays “ascending” or “descending” for each selected attribute. By default, return values are sorted in ascending order on the selected attributes. The user can click on the word “ascending” to change it to “descending” and vice versa.

The “Add” and “Remove” buttons are also accessible from the shared footer panel.

To add attributes to the Order By list, the user selects (clicks on) each attribute in the attribute tree view, and then clicks the “Add” button; or double-clicks the attribute in the tree. To remove attributes from the Order By list, the user selects them in the list and clicks the “Remove” button. To change the order of attributes, the user drags them upward or downward in the list.

After the user constructs a search, he/she can click on the Search button to execute the query and generate a tabular display of the search results. This causes the “Results” tab panel to be made visible. The objects (samples or genes) that satisfy the search conditions are displayed in a table, with the columns corresponding to the display attributes specified during search construction. The selection of attributes will be saved in the user's preferences.

The top of the panel indicates the total number of samples or genes, and the number that are currently selected.

The user can select one or more objects and perform an operation on them, such as saving them as a sample or gene set. Objects can be selected individually by clicking on their rows, or en masse by dragging over multiple rows; multiple discontiguous rows can be selected by control-clicking.

For simple attributes that are multi-valued, the multiple values will be concatenated together in a comma-separated string. Multivalued tuple attributes are shown in the form view only.

If the user wants to select different attributes for display, he/she can bring up the Select Display Attributes dialog from the View menu, and modify the display columns. This will cause GX Explorer to query for the new attributes only, without re-executing the entire search.

Samples or genes from different search windows can be combined in a single window, by selecting their rows in one window, copying them to the clipboard, and pasting them in the other window.

Adjacent to the tabular display, the sample and gene set windows each contain one or more form views that display selected attributes of one object at a time. Form views are more flexible than tabular displays, because more screen space can be provided to display complex attributes (such as simple and tuple multivalued attributes). They can be configured to use different types of widgets to display different attributes of the same type. For instance, a multivalued string attribute can be displayed as a comma-separated list, or as a multiline list of values. Multivalued tuple attributes can be displayed in tables. Attributes containing URLs that point to images (e.g., photomicrograph links for samples) can be displayed as images.

The form view for the main OPM class returned by the search—Biosample or Affy_item—is displayed in the right half of a split pane view, with the tabular display in the left pane. The views are coordinated, so that when the user selects a row in the tabular display, the corresponding object is displayed in detail in the form view. If multiple objects are selected in the tabular view, the first object selected is the one displayed in the form view.

If several form views are available, they are displayed in a tabbed pane, so that the user can select any one of them. The last form selected is saved in user preferences. If only one form view is available, there are no form tabs.

There are two kinds of form views: views generated automatically based on the OPM metadata for the associated class and views that are defined by configuration files, which are stored on the Gene Express application server. The configuration file specifies the attributes that are displayed, the types of fields used to represent them, and the layout of the fields. Configuration files are created and modified using a combination of a visual form editor tool and manual text editing.

A metadata-driven form view displays all attributes for an OPM object instance that have non-null values. The view has two main columns: a label column for the attribute names, and a value column for their values. Multivalued attributes have only one label, and the values are stacked one under the other. Each attribute label has an associated tooltip, that displays the attribute's description when the user holds the mouse cursor over the label. Tooltips are also provided for tuple or abstract rep attribute labels in table column headers.

Abstract attribute values are displayed using the rep attributes of the value class instance. Those with more than one rep attribute are displayed in a table, with columns corresponding to the rep attributes. Those with only one rep attribute are rendered like primitive attributes, i.e. with no columns or table headings. Abstract attributes with no rep attribute (which shouldn't exist), are rendered with their ID attribute value. For all types of abstract attributes, the value, or row of values is drawn in blue to indicate that the value text functions like an HTML link. When the user clicks on the value, the form is replaced with a form view for the value class. If the value class contains abstract attributes, these are also displayed as links that the user can click on to bring up yet another form, and so on. The metadata-driven form view contains left and right arrow buttons, so that the user can navigate backward or forward through the series of forms thus generated. The left and right arrow buttons are disabled when the user is at the beginning or end of the form stack, respectively. If the user selects a different instance in the tabular view, the form view is reset to the form for the Biosample or Affy_item class.

Some primitive attributes are also “activatable”; that is, the user can click on their value to get more information. Examples of attributes that have a custom activator, and custom format, are the “www” tuple components in the gene index. They are represented by the URL value, not the whole <a href=. . . > string which is the actual attribute value. The URL is preferred, because the label of the anchor is also stored in another component of the tuple that contains www attributes, so the URL gives more information. When the user clicks on a URL attribute, GX Explorer brings up the user's web browser and points it to the URL that is stored as the attribute value.

Tuple attributes, whether multivalued or not, are displayed in a table, whose columns correspond to the components of the tuple. The columns have headers that are the tuple component attribute names. If the user clicks on a cell that is not activatable, the whole tuple is displayed in a separate screen, as if it were a separate object, so that there is more space to display its components.

To create a Sample Set or a Gene Set based on the results from a search, the user first goes to the tabular display tab panel and selects the objects he/she wants to save; then chooses Save from the File menu. This brings up a “Save Data Object” dialog, in which the user can navigate to the folder where he/she wants to save the new Sample Set or Gene Set, and enter a name, description and permissions for it.

A paraphrase of the original query string (using display names for attributes instead of the internal names) will be logged as part of the “history” information for the object. If the samples have been manually selected or edited, this information will be logged also.

Users sometimes prefer to construct a sample or gene query and save it in the Workspace, to run at a later time. This is particularly true for sample searches, since new samples are added to the database frequently; users will want to see what samples matching their criteria have been added to the database since the last time they ran the query.

Thus, when a user reopens a SampleSet or GeneSet, the query conditions will be preset to those used when the sample/gene set was created. The user must be careful in this case when saving the results of the query to save to a new Sample Set if they don't want to obliterate the results from the previous search.

When a GeneSet is produced from an analysis operation such as GeneSignature, the query conditions will of course be empty.

Sample set searches look for objects of class GENOMICS in the sample (clinical) database. The search adds some conditions to the conditions specified by the user, so that only samples that have run through chips are returned. The genomics id of each sample is always displayed, and it is the number saved as the id of the sample, rather than the biosample id. A typical OQT query that is constructed by the sample search is shown below:

SELECT DISTINCT x, y.GENOMICS_ID,

x.BIOSAMPLE_ID,

x.SOURCE_ID,

x.BIOSAMPLE_ACCESSION_ID

FROM x IN BIOSAMPLE, y in FRAGMENT, g in GROUP_BASE

WHERE ((lower(x.ORGAN_NAME.ENOMEN)match“%colon%”))

AND y.GENOMICS_ID is not null

AND x.BIOSAMPLE_ID=y.BIOSAMPLE_ID

AND g.GENOMICS_ID=y.GENOMICS_ID

ORDER BY y.GENOMICS_ID;

Note the additional conditions that are added to the conditions specified by the user:

AND y.GENOMICS_ID is not null

AND x.BIOSAMPLE_ID=y.BIOSAMPLE_ID

AND g.GENOMICS_ID=y.GENOMICS_ID

Certain attributes will be selected in a custom way when specifying a condition for them.

The Genomics ID Query interface is intended for users who already know the genomics IDs for a group of samples they are interested in analyzing. It is particularly useful when the donor and/or clinical information for the samples has not yet been entered into the Sample Database. In this situation, the only information that is available about a sample is in the TARGET_TYPE and TARGET tables in the GX Data Warehouse.

The interface consists of a tabbed panel within the Sample Search window. The panel, labeled “Genomics ID Query”, contains a combo box, a text field and a field picker widget. The combo box lists all the target_type values found in the GX Data Warehouse, along with a special item labeled “All Target Types”. The field picker contains two multiselect list boxes, add, remove, delete and clear buttons. When the user makes a selection from the target_type combo box, the left hand field picker list box is updated to show all the genomics IDs for samples with the selected target type. The user can add genomics IDs to the list in the right hand list box by selecting them in the left hand list box and clicking the “Add” button, or simply by double-clicking them in the left hand list box. The user can remove IDs from the right hand list by double-clicking them, or by selecting them and clicking the Remove button.

The text field provides an alternate way to add genomics IDs to the list. The user can simply type in each genomics ID. When the user presses the Enter key after entering an ID, GX Explorer checks to make sure the ID is present (in the GXDW GROUP_BASE table), displays a warning dialog if not, and otherwise adds the ID to the selected genomics ID list.

The genomics ID query interface preferably does not allow external (non-GLGC) users to access private samples provided by other external customers. Therefore, ownership filtering is preferably performed on the list of genomics IDs from which the user can select, as well as on IDs that are typed in manually.

When the user is done selecting genomics IDs, he/she can go to the “Table Display” panel in the Sample Set window to view the attributes of the associated samples (if any information is available for them). The samples will initially all be selected. The user can create a Sample Set in the same way as with a normal sample search, by clicking the Save button in this panel and entering the Sample Set information in the Save Data Object dialog that will be displayed.

The Sample Set Import Utility allows a user to create a Sample Set based on a list of genomics IDs stored in a text file. In GX Explorer, this feature is integrated with the Genomics ID query interface. The Genomics ID panel is expanded to include an Import button. When the user clicks this button, a file open dialog is displayed; the user selects a file and clicks the Open button in the dialog, dismissing the dialog. GX Explorer parses the file into a list of genomics IDs, treating spaces, tabs, commas and newlines as delimiters. Any nonnumeric text is ignored. Each ID is checked against the GROUP_BASE table in the GX Data Warehouse, and if found, is added to the selected genomics ID list in the Genomics ID field picker. Any IDs that aren't found in the database are presented to the user in a warning dialog. Any IDs corresponding to private samples that aren't owned by the user are disallowed.

The Sample Set window will have menus for the following operations. In this context, “opening” a sample set means retrieving a sample set from the Workspace Manager. “Saving” a sample set means saving the sample set in the Workspace Manager. Each of these operations will display an Open or Save Data Object dialog, configured for opening or saving Sample Sets.

The New Window operation creates a new sample set window, so that the user can do two searches side by side and visually compare the results.

The Save Sample Set operation saves all samples from the search as a Sample Set.

The Save Selected Samples operation is the sSame as “Save Sample Set” , but will save only the samples that are selected.

The Open Sample Set operation will open a sample set and display it. The previously displayed sample set will be discarded. The resulting display of the opened sample set will function exactly like the display of a search results set, except that the Order By and the Conditions tab will have no effect. However, a user can then do another search and create a new sample set.

The Include Samples operation will open a sample set and append its samples to the current sample set, if they are not already in it. This effects a set union of sample sets.

The Exclude Samples operation will open a sample set, and remove any of its members that are found in the current sample set. This results in a set difference between the two sample sets.

The Intersect With operation will open a sample set and take its intersection with the current sample set. The resulting sample set will become the current sample set.

The Print operation prints a tabular representation of the current Sample Set. The Sample Set name and description are shown at the top of the report.

The History operation prints the “genealogical” information for the current Sample Set. Information displayed for Sample Set includes, bit is not limited to, sample set name and description, query conditions, query date, versions of SampleDB (schema and build), and log of operations performed, including set operations and manual editing.

The Sample Report operation displays a summary report of the samples currently loaded in the GX Data Warehouse.

A user creates a new Gene Set in multiple ways. In one approach, from the Genes menu in the GX Explorer menu bar, select “Gene Search”; this brings up a Gene Set window, with the “Gene Search” tab selected. The Gene Search interface is similar to the Sample Search interface, and is described in the next section.

In another approach, from the Genes menu in the menu bar, select “Sequence Search (BLAST)”; this brings up a Gene Set window with the “Sequence Search” tab enabled.

In yet another approach, select an existing GeneSet in the Navigator, duplicate it (by selecting Copy from the File menu or contextual menu, selecting another folder, and then pasting), and then open it. This brings up a Gene Set window with the tabular results display tab selected, listing the Affy gene fragments and their attributes in a table. The user can select genes and remove them from the Gene Set if he/she wishes, or can perform set operations on the Gene Set.

The Gene Search uses the generic OPM class search interface.

Chromosomes are preferably displayed in the query UI in numerical order, even though chromosome is a string attribute. X, Y and “mitochondria” preferably followss the numbered chromosomes.

Gene set searches look for objects of class Affy_fragment in the GX Index database. Many of the attributes in the gene index database contain HTTP links to external databases on the web. Custom form components will display these attributes appropriately, and provide a way for the user to browse the links (either by controlling an external Web browser, or embedding a Java browser component (such as the ICE browser).

The Sequence Search provides an alternative mechanism for selecting Affy fragments for constructing a Gene Set. It performs a BLAST homology search for a user-provided nucleotide or protein sequence, against a local database of GenBank sequences corresponding to the Affymetrix probe sets.

The interface is designed to be familiar to users who have run BLAST searches at NCBI or other sites. It consists of a tab panel, labeled “Sequence Search”, within the Gene Search window. The panel contains a combo box for selecting the BLAST variant to run (e.g. blastn for nucleotides, blastp for proteins, etc.); another combo box for selecting the sequence set to query (e.g. Human 42K set, mouse or rat chip set); a text field for entering additional options; and a scrollable text area for entering the sequence to query against (which may be pasted from the clipboard) in FASTA format.

The interface will also allow searching for multiple sequences at the same time. Users can paste in multiple sequences. They will be separated by the FASTA header lines, i.e. any line beginning with “>” will signify the start of a new sequence.

The interface preferably also supports the option of entering GenBank IDs and retrieving sequences, and allow sequences to be imported from local text files.

After the user enters the search parameters, he/she can click a “Search” button to run the homology search. When the search completes, GX Explorer shows the tabular display panel, listing the Affy fragments that match the input sequence, in descending BLAST score order. The table shows the user-selected attributes for each Affy fragment (retrieved from the user's preferences, or from the user's previous selections from the Select Display Attributes dialog); together with the score, P-value, and HSP values returned by BLAST for each matching fragment. The top of the panel shows the number of hits returned by the search.

From the tabular display panel, the user can select particular Affy fragments from the search results and save them as a Gene Set.

The Gene Set Import Utility allows a user to create a Gene Set based on a list of Affy probe set names and chip IDs stored in a text file. The sequence of operations is, in one embodiment, a process, as follows: (1) from the Gene Set window menu bar, the user selects the “Import Gene Set” menu item; (2) GX Explorer displays a file open dialog; the user selects a file and clicks the Open button in the dialog; the dialog is dismissed; (3) GX Explorer parses the file into a list of Affy fragment names and chip IDs, treating spaces, tabs, commas and newlines as delimiters. The entries in the file are in the same form as they are displayed by the analysis tools; for example, “M24899_at(5)” represents the instance of the gene fragment named “M24899_at” on chip 5 (the Hu6800 chip). Each ID is checked for validity against the GX Index database; and (4) GX Explorer queries the GX Index database for the user-selected return attribute values and displays the query results in the tabular display panel, with all Affy fragments initially selected. The user can then save the fragments as a Gene Set if he/she wishes.

The Gene Set window supports similar operations on Gene Sets as the Sample Set window does on Sample Sets.

The New Window operation creates a new gene set window, so that the user can do two searches side by side and visually compare the results.

The Save Gene Set operation saves all genes from the search as a Gene Set.

The Save Selected Genes operation is the same as “Save Gene Set”, but will save only the genes that are selected.

The Open Gene Query operation opens a Gene Query object from the Workspace and prepares the query interface for running the query (but does not actually run it).

The Open Gene Set operation will open a gene set and display it. The previously displayed gene set will be discarded. The resulting display of the opened gene set will function exactly like the display of a search results set, except that the Order By and the Conditions tab will have no effect. However, a user can then do another search and create a new gene set.

The Open Gene Query operation opens a Gene Query object from the Workspace and prepares the query interface for running the query (but does not actually run it).

The Include Genes operation will open a gene set and append its genes to the current gene set, if they are not already in it. This effects a set union of gene sets.

The Exclude Genes operation will open a gene set, and remove any of its members that are found in the current gene set. This results in a set difference between the two gene sets.

The Intersect With operation will open a gene set and take its intersection with the current gene set. The resulting gene set will become the current gene set.

The Print operation prints a tabular representation of the current Gene Set. The Gene Set name and description are shown at the top of the report.

The History operation prints the “genealogical” information for the current Gene Set. Information displayed for Gene Sets including, but not limited to, gene set name and description, query conditions, query date, versions of GX Index (schema and build), and log of operations performed, including set operations and manual editing.

If the Gene Set was exported from an analysis result, instead of query data display the genealogy information from the analysis object.

A Gene Signature analysis computes two sets of Affy fragments, given a Sample Set: those that are consistently present within the Sample Set, and those that are consistently absent. The user specifies the required consistency of expression as a pair of threshold percentages, one for the “present” set, the other for the “absent” set. The result can be thought of as a pair of Gene Sets. If there are 5 samples in the sample set, and the threshold percentages are set to 80% and 100%, respectively, then the “present Gene Set” of the Gene Signature contains those Affy fragments that are present in at least 4 out of 5 samples, while the “absent Gene Set” contains all Affy fragments that are absent in all samples.

Affy fragments that have “marginal” calls for a particular sample are treated the same as “absent” fragments. Fragments that have “unknown” calls are ignored in the Gene Signature computation. If, for a particular Affy fragment, p, m, and a are the numbers of samples for which the fragment was present, marginal, and absent, respectively, then the fractions p/(p+m+a) and (m+a)/(p+m+a) are computed; these fractions are compared against the present and absent threshold percentages to determine if the fragment belongs to either of the Gene Signature gene sets.

For example, suppose the Gene Express data warehouse contained the present/absent/marginal/unknown call values shown in the table below, for the sample set S={s1, s2, s3, s4} and the genes {g1, g2, g3, g4, g5, g6, g7, g8, g9}. (In reality there would be data for thousands of genes, but only nine genes are shown for illustration.) At the bottom of the column for each gene are shown the percentages computed from the numbers of present, absent and marginal calls for each gene across sample set S.

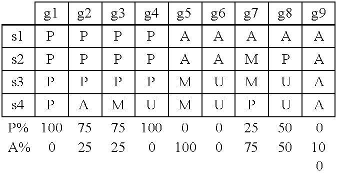

Suppose that the present and absent threshold percentages were both set to 75%. Then for this sample set, the Gene Signature operation returns a “present Gene Set” containing genes {g1, g2, g3, g4}, and an “absent Gene Set” containing {g5, g6, g7, g9}.

The Gene Signature analysis can also compute the mean, median, first and third quartile expression values, standard deviation and interquartile range for each gene in the present and absent sets. The user can select any or all of these values to be displayed in the Gene Signature report.

To compute a Gene Signature, a user begins by choosing Gene Signature from the Analysis menu. This brings up a frame window with a menubar (and maybe a toolbar), and multiple tabbed panels, labeled “Compute”, “GS Curve”, “Result Summary”, “Present Genes”, and “Absent Genes”. The “Compute” panel contains a form in which the user enters the threshold percentages, and selects a Sample Set to compute the signature over. The form contains a button labeled “Compute”; when the user clicks it, the “GS Curve” panel (described below) is displayed during the course of the computation. When the computation is complete, the “Result Summary” panel is displayed, summarizing the results of the analysis. At any time, the user can go back to the Compute tab, enter new parameter values, and recompute the signature.

The result summary panel displays the following information: (1) the name of the input sample set and the number of samples in it; (2) the number of gene fragments in the present and absent gene sets of the signature, along with the threshold percentages; and (3) a table listing the samples used to compute the signature, with links to the Sample Detail display for each sample. For each sample, the table shows the Affy chip types for which experiment data is available for the sample; the total numbers of present, absent and unknown calls; the numbers of present and unknown calls among the genes in the signature's present gene set; and the numbers of absent and unknown calls among the genes in the signature's absent gene set.

The “GS Curve” panel displays a pair of Gene Signature curves, one for the present gene set and one for the absent gene set. This display is designed to give the user a visual sense of whether the Sample Set is large enough to generate a valid gene signature. The curves are computed as follows:

Compute the present gene counts for each sample in the Sample Set.

Order the samples by present gene count in ascending order.

Initialize P to the set of present genes in the first sample. The height of the first point in the curve is the number of genes in P.

Intersect P with the set of present genes in the second sample, and repeat for each sample in the Sample Set. The heights of the successive points in the curve are the number of genes in P after each intersection step. The X axis component of each point is the index of the corresponding sample in the sorted Sample Set.

Repeat steps 1 through 4 for the absent genes, and plot the intersection set counts on a separate graph.

The height of each curve preferably decreases quickly at first and then level off. If the leveling off doesn't happen before there are no more samples, there probably aren't enough samples to produce a representative Gene Signature, and the user should consider constructing a larger Sample Set.

A Gene Signature computation can be saved in the Workspace Manager, and reloaded later on for further analysis and visualization. To save a Gene Signature, the user chooses the “Save” menu item in the Gene Signature window menu. (The Save menu item is disabled unless a signature has been previously computed or loaded.) GX Explorer displays a Save Data Object dialog, in which the user navigates to a project folder, enters a name and optional description for the Gene Signature, and sets the group and world access permissions.

The user can also save the present and absent gene sets of the signature as Gene Sets, by choosing the corresponding items from the Gene Signature window menu: “Save Present Gene Set” and “Save Absent Gene Set”. For either of these, GX Explorer presents a Save Data Object dialog configured for saving a Gene Set. The user is provided with an option to only include genes for which the median expression value, p-value, or frequency is above or below a user specified threshold.

There are two ways to load a previously saved Gene Signature object:

In the main GX Explorer window, select the object in the Workspace Navigator view and open it (using the menu or toolbar, or by double-clicking its icon). This brings up a new Gene Signature window, with the Result Summary panel displayed.

In an existing Gene Signature window, choose the Open menu item. GX Explorer displays an Open Data Object dialog, from which the user selects a signature object and clicks the Open button; this takes the user to the Result Summary panel. Any existing Gene Signature information for the window is discarded.

In the “Present Genes” and “Absent Genes” panels in the Gene Signature window, detailed expression data and selected gene attributes for the genes in the present and absent gene sets can be displayed in tabular form. Since the present and absent gene sets of a Gene Signature are usually quite large, numbering thousands of gene fragments, the user may want to sort the results so that interesting gene fragments are more likely to be displayed first. Thus, the Gene Signature View menu includes a submenu to select the attribute(s) and/or values to sort by. The rows can be sorted by gene attributes, by the average or median expression intensity (increasing or decreasing), or by a “frequency in database” value (defined as the fraction of samples among all samples for the same species in the GX Data Warehouse in which the gene is present). The default is to sort by decreasing order of median value.

The Options menu includes a pair of toggle items, “Use Affy Normalization” and “Use GLGC Normalization”; these function as a radio group so that only one or the other is checked. By default, Gene Logic-normalized expression values are used to compute the median and mean intensities and other statistics, but the user can select the Affy-normalized values instead if he/she wishes. The normalization option can be set independently for each Gene Signature window, so that results using different normalizations can be compared.

The View menu includes a “Select Display Attributes” item. If the user selects it, a dialog is displayed, similar to the Select Display Attributes dialog in the Gene Search interface, in which the user can select the attributes to be displayed for each Affy fragment. These default to the gene attributes stored in the user's preferences.

Gene attributes that refer to known gene or sequence cluster instances are displayed as links to detailed information about the gene or cluster.

The Options menu also includes toggle items labeled “Show Raw Expression Values” and “Show Raw Call Values”. When these are selected, the tabular detail displays show the expression values and/or present/absent calls for each sample used to compute the gene signature, displayed in one row for each gene in the present or absent set. Affy- or GLGC-normalized values are used, according to the user's normalization selection. These options are supported for export to Excel, Spotfire and S-Plus as well.

Other options for visualization include the Pathway and Chromosome Map displays, and export to Excel, Spotfire and S-Plus.

The History operation displays the “genealogical” information about the Gene Signature object (which doesn't change, once the object is created). This includes, but is not limited to, analysis parameters, date of analysis, and versions of RuntimeEngine matrix and databases used for analysis.

A Gene Signature Differential analysis compares the results of two Gene Signature analyses (which you must have previously computed and saved). Using the present and absent gene sets for the first and second Gene Signatures, the analysis derives four new sets of Affy fragments:

(1) Those that are in both the first Gene Signature's present gene set and the second's absent gene set.

(2) Those that are in both the first Gene Signature's absent gene set and the second's present gene set.

(3) Those that are in both present gene sets.

(4) Those that are in both absent gene sets.

In set language, these four gene sets are represented as:

P1∩A2

P2∩A1

P1∩P2

A1∩A2

For example, suppose there are two Gene Signature results with present and absent gene sets:

P1={1,2,3,4,5,11,12},A1={6,7,8,9,10,13,14}

P2={1,3,5,7,9,15,16},A2={2,4,6,8,10,17,18}

Then the outcome of the Gene Signature Differential is four intersection sets:

P1∩A2={2,4}

P2∩A1={7,9}

P1∩P2={1,3,5}

A1∩A2={6,8,10}

To compute a Gene Signature Differential, a user begins by choosing Gene Signature Differential from the Analysis menu. This brings up a frame window with multiple tabbed panels, labeled “Compute”, “Result Summary”, and “Visualize”. The “Compute” panel contains a form in which the selects the two Gene Signature objects to compare. The form contains a button labeled “Compute”; when the user clicks it, the “Result Summary” panel is displayed summarizing the results. At any time, the user can go back to the Compute tab, select different signature objects, and recompute the differential.

The result summary panel displays the following information: (1) the names of the two input Gene Signature objects, the thresholds used to compute them, and the sizes of their present and absent gene sets and (2) a table summarizing the numbers of gene fragments in the four intersection sets P1∩A2, P2∩A1, P1∩P2 and A1∩A2.

A Gene Signature Differential computation can be saved in the Workspace Manager, and reloaded later on for further analysis and visualization. To save a Gene Signature Differential, the user selects the “Save” menu item from the Gene Signature Differential window menu. The Save item is disabled unless a signature differential has been previously computed or loaded. GX Explorer displays a Save Data Object dialog, in which the user navigates to a project folder, enters a name and optional description for the Gene Signature Differential, and sets the group and world access permissions.

The user can also save any combination of the intersection sets of the differential as Gene Sets, by choosing the “Save Gene Set” item from the File menu. This brings up a Save Data Object dialog configured for saving a Gene Set, with checkboxes controlling which intersection sets are to be combined and saved: “Present in Both”, “Absent in Both”, “Present in 1 Only”, and “Present in 2 Only”.

There are two ways to load a previously saved Gene Signature Differential object:

In the main GX Explorer window, select the object in the Workspace Navigator view and open it (using the menu or toolbar, or by double-clicking its icon). This brings up a new Gene Signature Differential window, with the Result Summary panel displayed.

In an existing Gene Signature Differential window, select the Open menu item. This displays an Open Data Object dialog, from which the user selects a Signature Differential object. Any existing Gene Signature Differential data in the window is discarded.

The Gene Signature Differential window contains four panels in which detailed expression data and selected gene attributes for the genes in each intersection gene set are displayed in tabular form. The panels are labeled with descriptive names, e.g. “Present (Alzheimer's)+Absent(Normal)” for each intersection set. The P1∩P2 and A1∩A2 gene sets of a Gene Signature Differential are usually quite large, numbering thousands of gene fragments; the other intersection sets are generally much smaller, containing tens or at most hundreds of genes. For the larger intersection sets, the user may want to limit the number of rows displayed; for any of the sets, the user may want to sort the results so that interesting gene fragments are more likely to be displayed first. Thus, in addition to the table for the results display, the detail panels include a checkbox and text field to specify a maximum number of rows to display; and a combo box or other component to select the attribute(s) and/or values to sort by. The rows can be sorted by gene attributes, or by the average or median expression intensity (increasing or decreasing), or by a “frequency in database” value (defined as the fraction of samples among all samples for the same species in the GX Data Warehouse in which the gene is present).

The Options menu includes a pair of toggle items, “Use Affy Normalization” and “Use GLGC Normalization”; these function as a radio group so that only one or the other is checked. By default, Gene Logic-normalized expression values are used to compute the median and average intensities and other statistics, but the user can select the Affy-normalized values instead if he/she wishes. The normalization option can be set independently for each Gene Signature Differential window, so that results using different normalizations can be compared.

The View menu includes a “Select Display Attributes” item. If the user selects it, a dialog is displayed, similar to the Select Display Attributes dialog in the Gene Search interface, in which the user can select the attributes to be displayed for each Affy fragment. These default to the gene attributes stored in the user's preferences.

As with the Gene Signature, there is an option to display or export the raw expression and/or present/absent call values for each gene. In this case, however, there are two sets of expression values to display, one for each input sample set. The columns for each sample set are preferably thus grouped together, and the column label preferably indicates which sample set the associated sample belongs to.

Other options for visualization include the Pathway and Chromosome Map displays, and export to Excel, Spotfire and S-Plus.

A Fold Change Analysis computes, for each Affy fragment in the database, the ratios of the geometric means of the expression intensities between a control sample set and one or more experimental sample sets. The fold change is either this ratio or its reciprocal, if the ratio is less than one. The analysis categorizes Affy fragments by the fold change of their mean expression values between each pair of sample sets, and reports detailed expression information for those fragments whose fold changes fall within a user-specified range.

Confidence limits and p-values are also calculated when possible. The algorithm is based on a two-sided Welch modified two-sample t-test. It assumes that the logarithms of the expression intensities are distributed normally, which is a fairly good match to our data.

The null hypothesis used for the p-value computation is that the population means for the log(expression) values are the same between the two sample sets. The alternative hypothesis is that they are not. The confidence level for the difference in true means can be set by the user (it will default to a 95% confidence interval).

Both sample sets must have more than one sample. If one or both of the sample sets has only one member, then confidence limits and p-values cannot be calculated, though a fold change is still reportable using the algorithm described below.

Fold change is calculated on a per fragment basis: i.e., the following algorithm is applied to each fragment separately. Users have the option to choose Gene Logic-normalized or Affymetrix-normalized expression values for the analysis, but the same normalization must be used across all samples and genes.

For Gene Logic normalized expression values (“GL expression”) each chip has a standardized noise level of 10 (corresponding in concept to Q in the Affy scaling). More precisely, the distribution of the noise on each chip is estimated as part of the Gene Logic normalization and recalculate the expression levels so that the standard deviation for genes with zero expression is standardized to be 10.

For Affy normalized expression values, the actual noise value Q calculated for each chip experiment and stored in the GXDB database (under protocol_template ExpressionCallAbs, parameter_type Analysis, parameter_template ‘RawQ’) is used for the analysis.

The user also has the option to compute the fold change using only samples for each gene for which the gene is called present. When this option is selected, the numbers of samples nx and ny for each sample set will vary for different genes, and it may not be possible to compute p-values and confidence limits for every gene.

The inputs to the algorithm are 2 sample sets, X and Y, and 1 gene set; along with the user-specified confidence level CL% (between 0 and 100%, defaulting to 95%). For sample set X and a gene fragment in the gene set, the process is as follows:

If GLGC normalization is used, and GL expression <20 (i.e., 2*SD), set the expression value to 20. If Affy normalization is used, and Affy expression <2*Qmax, where Qmax=max(Qi) over all samples i where the gene is not called ‘unknown’ in both sample sets, set the expression value to 2*Qmax. If the user selected the option to use only samples where the gene was called present, and Affy normalization is used, Qmax is preferably also computed over just the samples where the gene was called present.

Given expression levels e(1), . . . , e(nX) across nx samples in sample set X, the logs: x(i)=1n(e(i)) are calculated

Calculate the mean(x), i.e., mean(x)=(sum over i of x(i))/nx

Calculate the variance(x), i.e., var(x)=(sum over i of (x(i)−mean(x))2)/(nx−1)

Repeat steps 1-4 for sample set Y.

Calculate a t statistic:

t=(mean(x)−mean(y))/s

where

s=sqrt( var(x)/nx+var(y)/ny)

If x and y come from normal populations, the distribution of t under the null hypothesis can be approximated by a t-distribution with (non-integral) degrees of freedom

DF=1/(c2/(nx−1)+((1−c)2)/(ny−1))

Where

c=var(x)/(nx*s2).

The calculation of the appropriate values depends on the cumulative T probability distribution function Pt(t, df) and its inverse tInverse(p, df), for which there are many available in numerous mathematical libraries.

Now the hypothesis FC=1 is equivalent to testing log FC=0, or mean(x)=mean(y). Therefore, calculate the p-value by:

Pval=Prob(|T|>t)=2*(1−Pt(t, DF))

where Pt(t, DF) is the cumulative T distribution with DF degrees of freedom and t is the statistic specified above.

Compute the fold change value and confidence limits. Given the user specified confidence level CL%, compute:

TI=s*tInverse((100+CL%)/200, DF)

Now the fold change and confidence interval can be calculated using:

μ=mean(x)−mean(y)

FC=exp(μ)

Lower CL%=exp(μ−TI)

Upper CL%=exp(μ+TI)

If FC<1, the fold change is 1/FC, and the direction is “lower”; otherwise the fold change is reported as FC and the direction as “higher”.

After computing the fold changes for each fragment between the control and experiment sample sets, the fragments are classified according to the range of fold change values. Typically the user is interested in all gene fragments that have fold changes greater than a certain value. Fragments for which all samples in both sample sets return an absent call may be included or excluded from the range classes.

To compute a Fold Change, a user begins by choosing Fold Change from the Analysis menu. This brings up a frame window with multiple tabbed panels, labeled “Compute”, “Summary”, and “Details”. The “Compute” panel contains a form in which the user selects a control Sample Set and one or more experiment Sample Sets to compute the fold change between. There is also a field in which the user can enter the confidence level CL% to use for computing confidence limits; the field value is initially set to 95. The form also has a checkbox, labeled “Use only samples where gene is present”, which is unchecked by default. If the user checks it, the fold change computation for each gene will only use expression values for samples where the gene is called present.

The form contains a button labeled “Compute”; when the user clicks it, the “Summary” panel is displayed showing the results summary. At any time, the user can go back to the Compute tab, select new Sample Sets, and recompute the fold change.

The result summary panel displays the following information: (1) the names of the input sample sets and the number of samples in each; (2) an indication of whether all samples were used to calculate the FC for each gene, or only the samples where the gene was present; and (3) a table listing the numbers of Affy gene fragments with fold changes in the following ranges: greater than 100, between 10 and 100, between 5 and 10, between 4 and 5, between 3 and 4, between 2 and 3, between 1 and 2, and with no change. The numbers are broken down by the direction of the change (higher and lower), and the totals are also shown. For each of these, the counts are given two ways: including all fragments, and excluding fragments for which all samples in both sample sets returned only absent calls. Fold change values computed on these latter fragments are less reliable, because they are based on low expression intensity values, for which the signal-to-noise ratio is low. When two or more experiment sample sets are analyzed, the columns are repeated, with columns for each experiment grouped together.

Instead of labeling columns/gene subsets/etc. with “Higher” and “Lower” directions, descriptions like “Higher in Melanoma samples” or “Higher in Normal liver” are employed.

A Fold Change computation can be saved in the Workspace Manager, and reloaded later on for further analysis and visualization. To save a Fold Change, the user selects the “Save” menu item from the Fold Change window menu. (The Save item is disabled unless a fold change has been previously computed or loaded.) GX Explorer displays a Save Data Object dialog, in which the user navigates to a project folder, enters a name and optional description for the Fold Change, and sets the group and world access permissions.

The Fold Change window menu also contains an item, “Save Gene Set”, through which the user can create a Gene Set of Affy fragments with fold changes in a specified range for one or more experiments. When the user selects this item, a dialog is displayed prompting the user for: (1) the range of fold change levels (if no upper value is given, then all genes with fold changes greater than the lower value are selected); (2) the direction (higher, lower or both); (3) the experimental sample set(s) for which the fold change must fall within or exceed the specified range. Preferably there is a checkbox option to control whether the fold change must be in or exceed the range for all samples selected, or at least one of them; and (4) whether or not to include genes for which all samples gave absent calls.

The dialog contains Save and Cancel buttons; when the user clicks the Save button, a Save Data Object dialog is displayed for the gene set.

There are two ways to load a previously saved Fold Change object:

In the main GX Explorer window, select the object in the Workspace Navigator view and open it (using the menu or toolbar, or by double-clicking its icon). This brings up a new Fold Change window, with the Result Summary panel displayed.

In an existing Fold Change window, select the Open menu item. This displays an Open Data Object dialog, from which the user selects a Fold Change object. Any existing Fold Change data in the window is discarded.

In the “Details” panel in the Fold Change window, fold change values, upper and lower confidence limits, p-values and selected gene attributes for genes with a selected range of fold change values can be displayed in tabular form. The panel includes the following components to determine which genes are displayed:

A pair of text fields in which the user enters the range of fold change levels (if no upper value is given, then all genes with fold changes greater than the lower value are selected).

A checkbox group (or multiselect list) with which the user specifies the experimental sample set(s) for which the fold change must fall within or exceed the specified range.

A checkbox to control whether the fold change must be in or exceed the range for all samples selected, or only one or more of them

A combo box with which the user selects the direction of fold changes to include genes for (higher, lower or both).

A checkbox with which the user indicates whether or not to include genes for which all samples gave absent calls.

The View menu includes a “Select Display Attributes” item. If the user selects it, a dialog is displayed, similar to the Select Display Attributes dialog in the Gene Search interface, in which the user can select the attributes to be displayed for each Affy fragment. These default to the gene attributes stored in the user's preferences.

In addition, the Details panel includes a combo box or other component to select the values to sort the output rows by. The genes can be sorted by gene attributes, or by the fold change value (increasing or decreasing), or by the “frequency in database” value (defined as the fraction of samples among all samples for the same species in the GX Data Warehouse in which the gene is present), or by the p-value. By default they are sorted by p-value.

An exemplary tabular report layout is presented below:

| | | SampleSet | SampleSet | SampleSet | Sample- | SampleSet |
|---|---|---|---|---|---|---|
| Affy | Sym- | 1 Lower | 1 Mean | 1 Upper | Set 1 P- | 2 Lower |
| name | bol | 95% CL | FoldChg | 95% CL | value | 95% CL |
| M28 | ALD- | .50 | 3.3 | 7.3 | .66 | 1.23 |
| 545_at | H3 | | | | | |

Optionally, the mean expression values for the two sample sets may be added as two extra columns. All the values for a gene fragment are displayed in one row. When multiple experiment sample sets are compared against the control sample set, additional groups of four columns are displayed for the fold change, confidence limits and p-value for each sample set.

As with the Gene Signature, there is an option to display or export the raw expression and/or present/absent call values for each gene. In this case, however, there are two or more sets of expression values to display, one for each input sample set. The columns for each sample set are preferably, thus, grouped together, and the column label preferably indicates which sample set the associated sample belongs to.

Other options for visualization include the Pathway and Chromosome Map displays, and export to Excel, Spotfire and S-Plus. It is worth noting though that, in the Pathway Map display, the user has the option of displaying fold change values for each gene fragment between the two sample sets, rather than expression intensities for each individual sample.

An Electronic Northern Analysis (or “E-Northern”) takes as input a user-defined Gene Set and one or more Sample Sets, and reports the range of expression levels for each Affy fragment in the Gene Set across each Sample Set, for all the samples where the Affy fragment is called present. The range is reported using percentile values, with the upper and lower percentile levels U% and L% specified by the user. If the user chooses U% to be 100 and L% to be 0, the analysis reports the maximum and minimum range of expression levels for all present calls; if the user chooses U%=75 and L%=25, the upper and lower quartile values are reported.

The E-Northern is computed as follows for each Sample Set:

For each Affy fragment in the user-specified Gene Set, count the number of Absent and Present calls across all samples for the given Sample Set. Obtain a score for the number of Absent and Present calls over the total number of samples. Omit samples with Unknown calls and do not include them in the total count of samples.

Marginal calls are grouped with Absent calls.

For each Affy fragment, sort the samples in which the fragment was called Present by ascending expression values. This generates a rank order R for each sample, R=1. . . N; where N is the number of samples with Present calls.

Calculate the rank order score, S=100*R/(N+1), for each sample for which the given Affy fragment was called Present.

Let U and L be the upper and lower percentile levels selected by the user. Find the largest rank order score less than or equal to U; the expression value for the sample with this score is reported as the upper U% percentile value. Find the smallest rank order score greater than or equal to L; the expression value for the sample with this score is reported as the lower L% percentile value.

For each Affy fragment, also report the Absent call score, Present call score and Median value. If there are an odd number of samples with Present calls for the fragment, the median value is the expression value for the sample with rank score 50; otherwise it is the average of the expression values for the two middle samples in the rank order.

For example: suppose the following data are provided for Affy fragment M12272_s_at(1):

| | | | | RANK |
|---|---|---|---|---|
| GENOMICS_ID | CALL | AVG_INTENSITY | RANK | SCORE |
| 171 | a | −103 | NA | NA |
| 195 | a | −99 | NA | NA |
| 189 | a | −49 | NA | NA |
| 148 | m | −43 | NA | NA |
| 157 | m | −34 | NA | NA |
| 135 | u | 0 | NA | NA |
| 199 | u | 0 | NA | NA |
| 144 | p | 20 | 1 | 5.56 |
| 177 | p | 25 | 2 | 11.11 |
| 292 | p | 29 | 3 | 16.67 |
| 104 | p | 47 | 4 | 22.22 |
| 145 | p | 55 | 5 | 27.78 |
| 133 | p | 63 | 6 | 33.33 |
| 141 | p | 78 | 7 | 38.89 |
| 142 | p | 78 | 8 | 44.44 |
| 152 | p | 91 | 9 | 50.00 |
| 180 | p | 97 | 10 | 55.56 |
| 134 | p | 109 | 11 | 61.11 |
| 173 | p | 111 | 12 | 66.67 |
| 124 | p | 133 | 13 | 72.22 |
| 97 | p | 146 | 14 | 77.78 |
| 158 | p | 149 | 15 | 83.33 |
| 132 | p | 256 | 16 | 88.89 |
| 156 | p | 502 | 17 | 94.44 |

The data are shown with the samples with Present calls sorted by rank order and the rank scores computed in the last column.

To compute a E-Northem, a user begins by choosing E-Northem from the Analysis menu. This brings up an E-Northem window, which is a frame window with a menu bar and multiple tabbed panels, labeled “Parameters”, “Results”, and “Visualization”.

The “Parameters” panel contains a form in which the user selects one or more Sample Sets and a Gene Set, and enters the percentile values. The user chooses Sample Sets from a field picker style component, containing a Workspace Navigator tree view on the left and a list box on the right. The tree view is configured to display Sample Sets only. The field picker also contains the usual Add, Remove, Clear, up-arrow and down-arrow buttons. The arrow buttons are used to change the order of the sample sets in the list box; this will be the order in which the sample sets are displayed in the tabular E-Northern results.

Sample sets from different species cannot be mixed in an E-Northern analysis; the interface will detect a species mismatch and prevent the sample set from being added to the list.

The user enters the percentile levels through text fields, whose values are defaulted to 75% and 25% for the upper and lower percentiles respectively. Values greater than 100 or less than 0 cannot be entered.

The form also contains a checkbox controlling whether Marginal calls are grouped with Present or Absent calls.

The Options menu includes a pair of toggle items, “Use Affy Normalization” and “Use GLGC Normalization”; these function as a radio group so that only one or the other is checked. By default, Gene Logic-normalized expression values are used to compute the upper and lower percentiles and other derived statistics, but the user can select the Affy-normalized values instead if he/she wishes. The normalization option can be set independently for each E-Northern window, so that results using different normalizations can be compared.

The parameter form supports several ways of selecting the Gene Set:

The user can click a “Browse Gene Sets” button, which brings up an Open Data Object dialog, configured to display all preexisting gene sets the user has read access to; the user can make his/her selection from this dialog.

The user can select a checkbox labeled “Use all genes for species”.

The user can click a button labeled “Use genes specific to Sample Sets”. This brings up a new dialog window containing a multiselect list box listing the Sample Sets selected on the parameter form, and a pair of text fields for entering the Gene Signature present call threshold percentage. The threshold is defaulted to 50%. After the user selects one or more Sample Sets and enters the threshold value and clicks the OK button to dismiss the dialog, GX Explorer constructs two “super-Sample Sets” from the selected and unselected sample sets, computes Gene Signatures for these two “super Sample Sets”, and then computes a Gene Signature Differential for the two signatures. The Gene Set used for the E-Northem computation is then the set of genes in the “present” set of the Gene Signature of the selected samples, intersected with the “absent” set of the Gene Signature of the unselected samples.

The form contains a button labeled “Compute”; when the user clicks it, the “Results” panel is displayed showing the results table. At any time, the user can go back to the Parameters tab, enter new parameter values, and recompute the E-Northem.

An Electronic Northern analysis can be saved in the Workspace Manager, and reloaded later on to be visualized or exported. To save an E-Northern, the user selects the “Save” menu item from the an E-Northern window menu. (The Save item is disabled unless an E-Northern has been previously computed or loaded.) GX Explorer displays a Save Data Object dialog, in which the user navigates to a project folder, enters a name and optional description for the E-Northern, and sets the group and world access permissions.

There are two ways to load a previously saved E-Northern object:

In the main GX Explorer window, select the object in the Workspace Navigator view and open it (using the menu or toolbar, or by double-clicking its icon). This brings up a new E-Northern window, with the Result Summary panel displayed.

In an existing E-Northern window, select the Open menu item. This displays an Open Data Object dialog, from which the user selects an E-Northern object. Any existing E-Northern data in the window is discarded.

There are various possible tabular output formats for the E-Northern results:

Columns are grouped in 5's. Each group is spanned by the name of the Sample Set. Each subgroup of 5 has the headings as above: Absent Score, Present Score, Lower L%, Median and Upper U%.

The rows are labeled with the names of the Affy Fragments.

Users can also request extra columns beside the Affy Item column to display gene attributes, such as gene symbols or sequence cluster names. They can do this through the “Select Display Attributes” item in the View menu. If the user selects it, a dialog is displayed, similar to the Select Display Attributes dialog in the Gene Search interface, in which the user can select the attributes to be displayed for each Affy fragment. These default to the gene attributes stored in the user's preferences.

In a second alternative columns are headed by the name of each Sample Set. Rows are grouped in 5's, each group of 5 is spanned by the name of the gene. The rows have the same names as specified in Alternative 1. This format is easier to read across tissues because it has fewer columns.

A horizontal box and whiskers style plot, for one gene at a time, with sample sets on the Y axis and expression ranges (on a log scale) on the X axis may also be supported. This could be paired with a horizontal bar chart aligned with the sample sets, showing the percentage of present calls for the gene for each sample set. The user will have to select a gene in the tabular display and go to another panel to see the graphical display. The sample sets will be arranged in decreasing order of median expression value for the gene.

The Expression Data Tool provides users with a way to display and visualize expression data directly, for the Affy fragments in a selected Gene Set, over the samples in one or more Sample Sets. The data may be displayed in tabular format within the Expression Data Tool window, in a Pathway or Chromosome Map visualization, or may be exported to any of the external tools supported by GX Explorer. Users can display either individual expression values, or aggregate values computed from them, such as means, medians and standard deviations.

The Expression Data Tool is invoked by selecting it from the Analysis menu in the main GX Explorer window. This creates an Expression Data Tool window. The window has two tabbed panels, one for selecting the Gene Set and Sample Set(s), the data to be displayed and the format, the other for displaying the results.

The Gene Set may be selected either by choosing it from a single-select Navigator tree view, or by choosing a pathway (which implicitly defines a Gene Set, consisting of all the genes involved in the pathway). The Sample Sets are selected from a separate Navigator tree.

There are various options for the type and format of data displayed:

Individual expression values in “tall skinny” format. The user can request that expression intensity values and/or present/absent calls be displayed, together with selected gene and sample attributes. One set of values for a gene/sample pair is displayed in each row of the output.

Individual expression values in “short fat” format. The user can request either intensity values or present/absent calls (but not both), together with selected gene attributes and sample attributes. The expression values and gene attributes are displayed on one row for each gene. The sample attributes are concatenated and displayed in the column headers.

Aggregate values in “tall skinny” format. This option is used with grouped data (such as toxicology studies), where each input Sample Set corresponds to a study group. With this option, one row of values is displayed for each gene/Sample Set combination. The user may select one or more sample attributes, and one or more aggregate values to display for each gene for each Sample Set; the sample attribute(s) chosen must have the same value over all the samples in each Sample Set, or an “NA” value will be displayed in the corresponding column of the output. If no common sample attribute is selected, the Sample Set name is displayed. The aggregate values that may be computed for and displayed for each Sample Set include, but are not limited to, mean expression intensity, median expression intensity, standard deviation of expression intensity, upper and lower quartiles of expression intensity, interquartile range of expression intensity, percentage of present calls, and p-values for present calls.

Aggregate values in “short fat” format. The user can select the same values and attributes as for the previous option, but in this case the values and attributes for each gene are displayed in one row. The user may select one or more sample attributes, which must have the same value over all the samples in each Sample Set; these attributes are concatenated to generate the column headers for the aggregate expression values.

Thus, the parameters panel preferably have a control to select the type of data displayed (aggregate or individual values), and a control to select the format (“tall skinny”=one row per data point, or “short fat”=one row per Affy gene fragment). When aggregate data is selected, a set of controls is enabled so that the user can select the aggregate values to be displayed from the list above.

To see the results, the user goes to the Results panel. This shows the first few dozen rows of data in a tabular display, in the format selected by the user in the parameters panel. Because the number of rows of data may be huge, GX Explorer needs to be intelligent in transferring data from the Runtime Engine, so that the user does not have to wait for all the data to be downloaded over the network. The following design criteria preferably are applied to tabular displays in all windows where large amounts of data may be displayed: (1) rows are preferably transferred in blocks of a few hundred at a time, (2) the scrollbar thumb size is preferably scaled to the actual number of rows of data, so that the user can jump scroll to the middle or end of the data set, (3) when the user scrolls to the middle or end of the data set, GX Explorer preferably transfers the block of data that is to be made visible without first transferring the intervening blocks, and (4) data transfers are preferably done in a separate thread from the UI thread, so that the user does not have to wait for data transfer to complete before the display repaints or responds to user actions.

The standard visualization and export options are supported, through Visualization, Export and Invoke menus. Note that exports to Spotfire and S-Plus require tall-skinny and short-fat formats, respectively; exports to Excel or plain text can be done in either format.

Virtually anywhere in the GX Explorer interface that tabular data is displayed, there is an option for exporting the data to external applications such as Excel, Spotfire or S-Plus. Each of the main application window types—Sample Set, Gene Set, Gene Signature, Gene Signature Differential, Fold Change, E-Northern, Expression Data, and Cluster Analysis—has Export and Invoke menus, with items for each supported application, which are enabled when a tabular display is visible. The user can either select rows to be exported, or choose to export everything.

To export data to a local file, in the appropriate format for an application, the user selects the data to be displayed and the format to display it in, goes to the tabular display panel, optionally selects rows to export, and chooses the application to export to from the Export menu. GX Explorer displays a Save File dialog, where the user specifies the file to save to.

To invoke the application directly, without going through the save step first, the user chooses the application from the Invoke menu instead. The data is saved automatically to a temporary file; then GX Explorer runs the application with the file as input.

A pathway visualization is a flowchart graph of the components of a metabolic or signalling pathway, highlighted with colored bands to denote the expression levels of the genes or gene products involved in the pathway. The bands may be divided horizontally into separate rectangles, each corresponding to an expression level for a particular sample. Alternatively, the pathway visualization may be used in conjunction with a Fold Change analysis, with the band colors corresponding to fold change values.

In a metabolic pathway, the components are boxes representing enzymatic activities (identified by EC numbers). Strongly and weakly expressed genes encoding enzymes are darkly and lightly shaded, respectively. Multiple genes may code for enzymes with the same activity (e.g., the many different alcohol dehydrogenases), and multiple Affy fragments may represent the same gene; in this case multiple bands are drawn for each enzyme, one for each Affy fragment. The underlying pathway diagrams are obtained from KEGG.

In a signalling or regulatory pathway, the nodes in the graph may represent specific genes or gene products, although enzymes may be present as well. As before, multiple Affy fragments may probe for the same gene, so multiple bands may be drawn for each node.

Pathway visualizations are typically performed for a particular sample set and gene set. The gene set may be computed indirectly from sample sets(s) using the Gene Signature, Gene Signature Differential or Fold Change Analysis tools, or may be selected directly from the Expression Output tool. Typically a gene set overlaps with several pathways, and the user selects the pathway of interest.

In a future version of GX Explorer, there will be a mechanism for editing pathway maps, so that user-defined pathways can be supported.

Pathway visualizations are available as an option for the Gene Signature, Gene Signature Differential, Fold Change Analysis, and Expression Output tools, by selecting “Pathway Map” from the Visualization menu in the tool-specific window. This menu item is disabled unless the user has run or opened an analysis in that window. GX Explorer displays a separate window, with three tabbed panels: “Gene Set Selection”, “Pathway Selection”, and “Display”.

The Gene Set Selection panel is tool-specific; it provides controls for the user to select the genes whose expression values will be visualized. For Gene Signature, the user can select the present and/or absent gene sets; for a Gene Signature Differential, one or more of the four intersection sets; for Fold Change, genes with fold changes in a specified range; for Expression Output, any existing Gene Set in the Workspace Manager.

The Pathway Selection panel is the same for all tools, except Fold Change. It displays a list of pathways whose genes overlap with the selected Gene Set, sorted in descending order of the number of overlapping Affy fragments. For each pathway, there are buttons corresponding to the following three options:

Median: Display the median expression levels for each Affy fragment in the selected Gene Set that overlaps the pathway, over all samples in the input Sample Set.

Raw: Display the raw expression levels for each Affy fragment in the selected Gene Set that overlaps the pathway, over all samples in the input Sample Set.

All: Display the raw expression levels for all Affy fragments that map to the pathway, regardless of the user's Gene Set selection, over the samples in the input Sample Set.

For Fold Change analysis, there is an additional Fold Change option, which displays the fold change values for each Affy fragment in the selected Gene Set that overlaps the pathway.

When the user selects one of these options, the Display panel is displayed. This panel contains a split pane view. The left hand pane gives a tabular summary of the genes and expression values shown in the display. The right hand pane contains the actual Pathway Map. The splitter can be moved, so the user can see more or less of the map or the table.

When many Affy fragments map to an enzyme or other component of a pathway, the colored bands can be hard to distinguish visually; they may also obscure the EC number, gene symbol or other identifier for the component. To get summary information about a pathway component, the user can just move the mouse cursor over it; a tooltip is displayed showing the enzyme or gene name. To get more detailed information, the user can click on the component. This brings up or updates the contents of a separate window with a tabular display similar to that in the left pane of the Display panel, but restricted to Affy fragments that map to the selected component. The two windows are separated on the user's screen as much as possible, taking advantage of multiple monitors if available, so that both the pathway map and the detail display can be visible simultaneously.

The pathway map view also contains a zoom control, so the user can magnify parts of the pathway. This may make the magnified GIF image look bitmappy, but it also allows more room to display the colored bands.

A chromosome map visualization is a display that shows the expression levels of Affy fragments (selected from an analysis as described in the next section) aligned with a map showing their chromosomal locations. The display allows users to select a subregion of a chromosome, providing a higher resolution view of the fragment locations.

The chromosome display is currently implemented for human chromosome 22 only. When sufficiently complete sequence data is published for other chromosomes, of human and other species, those chromosomes will be supported as well.

The chromosome map shows an ideogram for a single selected chromosome. A vertical bar is shown next to the ideogram, with a pair of diagonal lines connecting the ends of the bar to a pair of handles on the ideogram. The user can drag these handles to select a region of the chromosome to be visualized in detail.

The expression levels of the Affy fragments mapped to the selected region are displayed as colored bands on either side of the vertical bar, according to which strand the fragment is mapped to. The color and length of the bands correspond to the expression level. The user can also choose to show the presence or absence of the Affy fragment only; with this option, all the bands have the same color and length, and are only displayed for Affy fragments that are called present.

The names of the Affy fragments are shown next to the bands. The user can move the cursor over the fragment name to see gene or cluster attributes associated with the fragment (either as tooltips or in a separate window).

Chromosome map visualizations are available as an option for the Gene Signature, Gene Signature Differential, Fold Change Analysis, and Expression Data tools, by selecting “Chromosome Map” from the Visualization menu in the tool-specific window. This menu item is disabled unless the user has run or opened an analysis in that window.

The gene fragments displayed in the chromosome map, and how they are displayed, depends on the analysis type:

For Gene Signature, the genes in the Present set are shown. Either the mean or median expression value across the input sample set may be used to color the bars.

For Gene Signature Differential, the user can select either or both of the “present in one/absent in the other” intersection sets. The mean or median expression values over the sample set in which the gene is present may be used to color the bars. When two intersection sets are selected, two columns of bars are displayed; a legend indicates which column corresponds to which intersection set.

For Fold Change, the user can select genes with fold changes in a specified range.

For Expression Data, the genes are those in the selected Gene Set that are present in some user specified percentage of samples in the selected Sample Set.

Configuration files for the databases will be stored on the server machine and downloaded to the client machine through HTTP, or some other network protocol. The user won't have to upgrade the client software to see a new form, a new attribute, among others.

With regard to the Implementation Overview, the Gene Express user interface is built in Java, using CORBA to communicate with a Runtime Engine and with the sample, GX Index, and workspace management servers. The Runtime Engine will handle all compute- and memory-intensive operations, such as clustering, computation of Gene Signatures, and fold change analysis.

The Java interface will use the JFC/Swing widget set, for performance and extensibility.

Various preferred embodiments of the invention have been described in fulfillment of the various objects of the invention. It should be recognized that these embodiments are merely illustrative of the principles of the invention. Numerous modifications and adaptations thereof will be readily apparent to those skilled in the art without departing from the spirit and scope of the present invention.

1. A method of integrating multiple gene databases containing gene expression data, gene annotation data, and sample data, the method comprising:
defining new classes and associations for the gene expression data, gene annotation data, and sample data contained in at least one of the multiple gene databases;
grouping gene fragments into at least two groups; and
annotating gene fragments,
so as to permit a database analytical engine to analyze the gene expression data, gene annotation data contained in the multiple gene databases.

2. The method of integrating multiple gene databases containing gene expression data, gene annotation data, and sample data according to claim 1, wherein the at least two groups are known and not known.

3. The method of integrating multiple gene databases containing gene expression data, gene annotation data, and sample data according to claim 1, wherein the annotating if gene fragments comprises automatic annotation of predefined fields.

4. The method of integrating multiple gene databases containing gene expression data, gene annotation data, and sample data according to claim 1, wherein the annotating if gene fragments comprises manual annotation of individual gene fragments.

5. The method of integrating multiple gene databases containing gene expression data, gene annotation data, and sample data according to claim 2, wherein the grouping of gene fragments into at least two groups comprises name-based matching.

6. The method of integrating multiple gene databases containing gene expression data, gene annotation data, and sample data according to claim 2, wherein the grouping of gene fragments into at least two groups comprises sequence-based matching.

7. The method of integrating multiple gene databases containing gene expression data, gene annotation data, and sample data according to claim 2, wherein the grouping of gene fragments into at least two groups comprises manual data curation.

8. A computer program product comprising a computer-usable medium having computer-readable program code embodied thereon relating to multiple gene databases containing gene expression data, gene annotation data, and sample data, the computer program product comprising computer-readable program code for effecting the following steps within a computing system:
defining new classes and associations for the gene expression data, gene annotation data, and sample data contained in at least one of the multiple gene databases;
grouping gene fragments into at least two groups; and
annotating gene fragments,
so as to permit a database analytical engine to analyze the gene expression data, gene annotation data contained in the multiple gene databases.

9. The computer program product comprising a computer-usable medium having computer-readable program code embodied thereon relating to multiple gene databases containing gene expression data, gene annotation data, and sample data according to claim 8, wherein the at least two groups are known and not known.

10. The computer program product comprising a computer-usable medium having computer-readable program code embodied thereon relating to multiple gene databases containing gene expression data, gene annotation data, and sample data according to claim 8, wherein the annotating if gene fragments comprises automatic annotation of predefined fields.

11. The computer program product comprising a computer-usable medium having computer-readable program code embodied thereon relating to multiple gene databases containing gene expression data, gene annotation data, and sample data according to claim 8, wherein the annotating if gene fragments comprises manual annotation of individual gene fragments.

12. The computer program product comprising a computer-usable medium having computer-readable program code embodied thereon relating to multiple gene databases containing gene expression data, gene annotation data, and sample data according to claim 9, wherein the grouping of gene fragments into at least two groups comprises name-based matching.

13. The computer program product comprising a computer-usable medium having computer-readable program code embodied thereon relating to multiple gene databases containing gene expression data, gene annotation data, and sample data according to claim 9, wherein the grouping of gene fragments into at least two groups comprises sequence-based matching.

14. The computer program product comprising a computer-usable medium having computer-readable program code embodied thereon relating to multiple gene databases containing gene expression data, gene annotation data, and sample data according to claim 9, wherein the grouping of gene fragments into at least two groups comprises manual data curation.
